(19) United States
(12) Patent Application Publication
(10) Pub. No.: US 2023/0071410 A1
(43) Pub. Date: Mar. 9, 2023

(54) BIOLOGICAL INFORMATION MEASUREMENT APPARATUS AND NON-TRANSITORY COMPUTER READABLE MEDIUM

(71) Applicant: FUJIFILM Business Innovation Corp., Tokyo (JP)

(72) Inventors: Hideyuki UMEKAWA, Kanagawa (JP); Kazuhiro SAKAI, Kanagawa (JP); Hideaki OZAWA, Kanagawa (JP); Manabu AKAMATSU, Kanagawa (JP)

(73) Assignee: FUJIFILM Business Innovation Corp., Tokyo (JP)

(21) Appl. No.: 17/712,573

(22) Filed: Apr. 4, 2022

(30) Foreign Application Priority Data
Sep. 7, 2021 (JP) 2021-145493
Sep. 16, 2021 (JP) 2021-151277

(51) Int. Cl. A61B 5/00; A61B 5/1455; G16H 10/40

(52) U.S. Cl. CPC A61B 5/486; A61B 5/14551; A61B 5/4884; G16H 10/40

(57) ABSTRACT

A biological information measurement apparatus includes a processor configured to: if a predetermined number of plural measurements of an oxygen circulation time are to be performed, before a predetermined oxygen-circulation-time measurement period ends during a first measurement of the oxygen circulation time, notify a test subject of a breath-hold instruction as a preparation for a second measurement of the oxygen circulation time, the test subject being a person for whom the oxygen circulation time is measured, the second measurement being a subsequent measurement to the first measurement.

### CROSS-REFERENCE TO RELATED APPLICATIONS

This application is based on and claims priority under 35 USC 119 from Japanese Patent Application No. 2021-145493 filed Sep. 7, 2021, and Japanese Patent Application No. 2021-151277 filed Sep. 16, 2021.

### BACKGROUND

### (i) Technical Field

The present disclosure relates to a biological information measurement apparatus and a non-transitory computer readable medium.

### (ii) Related Art

Japanese Unexamined Patent Application Publication No. 2019-166147 discloses a biological information measurement apparatus including a notification unit that, at a prescribed time that prescribes a breath-hold period, gives a resumption notification that instructs a test subject, who is holding their breath, to resume breathing, and a measurement unit that measures an oxygen circulation time representing a time for oxygen taken into the test subject's body to reach a predetermined body part due to resumption of breathing of the test subject.

Japanese Unexamined Patent Application Publication No. 2006-231012 describes an oxygen transport circulation time measurement method that calculates a change in oxygen saturation on the basis of an arterial blood absorbance signal extracted from a living body using a sensor. The oxygen transport circulation time measurement method changes the amount of oxygen inhaled by the living body and also measures the time from a reference point until a change in the oxygen saturation in arterial blood, the reference point being a point in time at which the amount of oxygen inhaled by the living body is changed.

Japanese Unexamined Patent Application Publication No. 2019-166144 discloses a biological information measurement apparatus that can measure a change in a blood oxygen concentration. The biological information measurement apparatus includes a correction unit that receives a first signal representing a change in the amount of light of a first wavelength detected from a living body and a second signal representing a change in the amount of light of a second wavelength detected from the living body, and corrects at least one of the first signal and the second signal to reduce a difference between the amount of change in the first signal and the amount of change in the second signal associated with a change in the amount of arterial blood of the living body, and a calculating unit that calculates a change in the blood oxygen concentration in the living body on the basis of the first signal and the second signal of which at least one is corrected by the correction unit.

### SUMMARY

By using a biological information measurement apparatus, the oxygen circulation time has been measured plural times as follows. First, a change in oxygen saturation in blood is measured until a predetermined measurement time ends, and then, a subsequent measurement is performed. Thus, in order to measure the oxygen circulation time three times, the time [time for measuring oxygen circulation time once×3] has been necessary.

Aspects of non-limiting embodiments of the present disclosure relate to a biological information measurement apparatus that can shorten the time for measuring the oxygen circulation time plural times as compared with a case where, after the predetermined measurement time of the oxygen circulation time ends, a subsequent measurement of the oxygen circulation time starts, and a non-transitory computer readable medium.

The simplest way to change the test subject's blood oxygen concentration is that the test subject holds their breath. However, for the uncomfortableness of holding their breath, they may hold their breath with much air inhaled in their lungs. In this case, a waveform pattern representing the change in the blood oxygen concentration becomes inappropriate, from which an inflection point in the waveform pattern generated by the change in the blood oxygen concentration is not to be specified.

The existing technology has required a complex algorithm in order to determine whether the waveform pattern is appropriate, imposing a heavy burden on the determination processing. Thus, a simple determination method has been desired.

Aspects of non-limiting embodiments of the present disclosure relate to a biological information measurement apparatus that may determine that a waveform pattern is appropriate in a more simplified manner than in a case of determining whether the waveform pattern is appropriate by using an existing algorithm, and a non-transitory computer readable medium.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided a biological information measurement apparatus including a processor configured to: if a predetermined number of plural measurements of an oxygen circulation time are to be performed, before a predetermined oxygen-circulation-time measurement period ends during a first measurement of the oxygen circulation time, notify a test subject of a breath-hold instruction as a preparation for a second measurement of the oxygen circulation time, the test subject being a person for whom the oxygen circulation time is measured, the second measurement being a subsequent measurement to the first measurement.

### BRIEF DESCRIPTION OF THE DRAWINGS

Exemplary embodiments of the present disclosure will be described in detail based on the following figures, wherein:

FIG. 1 schematically illustrates an example of measuring oxygen saturation in blood;

FIG. 2 is a graph illustrating an example of changes in the amount of light absorbed by a living body;

FIG. 3 illustrates an example of amounts of light absorbed by oxyhemoglobin and deoxygenated hemoglobin at different wavelengths;

FIG. 4 is a diagram illustrating a configuration example of a biological information measurement apparatus;

FIG. 5 illustrates an arrangement example of light emitting elements and a light receiving element;

FIG. 6 illustrates another arrangement example of the light emitting elements and the light receiving element;

FIG. 7 illustrates an example of changes in the oxygen saturation in the blood;

FIG. 8 is a diagram illustrating a configuration example of a principal portion in an electrical system of the biological information measurement apparatus;

FIG. 9 is a first half of a flowchart illustrating an example of a flow of a biological information measurement process according to a first exemplary embodiment and a second exemplary embodiment;

FIG. 10 is a second half of the flowchart illustrating the example of the flow of the biological information measurement process according to the first exemplary embodiment;

FIG. 11 is a graph illustrating a temporal change example of the oxygen saturation of a test subject obtained through the biological information measurement process according to the first exemplary embodiment;

FIG. 12 is a graph illustrating a temporal change example of the oxygen saturation of the test subject in a case where a first LFCT measurement and a second LFCT measurement are not performed in an overlapping manner;

FIG. 13 is a second half of the flowchart illustrating the example of the flow of the biological information measurement process according to the second exemplary embodiment;

FIG. 14 is a graph illustrating a temporal change example of the oxygen saturation of the test subject in a case where an inflection point of the oxygen saturation of the test subject is detected before a predetermined over-time observation time during a final LFCT measurement elapses;

FIG. 15 is a graph illustrating a temporal change example of the oxygen saturation of the test subject in a case where the inflection point of the oxygen saturation of the test subject is detected after the over-time observation time during the final LFCT measurement elapses;

FIG. 16 illustrates an example of a relationship between the inflection point of the oxygen saturation and a reference value of the oxygen saturation;

FIG. 17 is a graph illustrating an example of changes in the amount of light received from light reflected from the living body according to a third exemplary embodiment;

FIG. 18 is a schematic diagram for describing a Doppler shift produced in a case where blood vessels are irradiated with laser light according to the third exemplary embodiment;

FIG. 19 is a schematic diagram for describing a speckle produced in a case where a blood vessel is irradiated with laser light according to the third exemplary embodiment;

FIG. 20 is a graph illustrating an example of a spectral distribution by frequency in a unit time according to the third exemplary embodiment;

FIG. 21 is a graph illustrating an example of changes in an amount of blood flow per unit time according to the third exemplary embodiment;

FIG. 22 is a schematic diagram for describing a principle of measuring a respiration waveform according to the third exemplary embodiment;

FIG. 23 is a schematic diagram for describing a principle of measuring an output according to the third exemplary embodiment;

FIG. 24 is a graph for describing an example of a method of measuring an LFCT according to the third exemplary embodiment;

FIG. 25 is a block diagram illustrating an example of an electrical configuration of a biological information measurement apparatus according to the third exemplary embodiment;

FIG. 26 illustrates an arrangement example of light emitting elements and a light receiving element in the biological information measurement apparatus according to the third exemplary embodiment;

FIG. 27 illustrates another arrangement example of the light emitting elements and the light receiving element in the biological information measurement apparatus according to the third exemplary embodiment;

FIG. 28 is a graph illustrating an example of sampling timings of data in the light receiving element according to the third exemplary embodiment;

FIG. 29 is a block diagram illustrating an example of a functional configuration of the biological information measurement apparatus according to a third exemplary embodiment;

FIG. 30A is a scatter diagram illustrating an example of a correlation between an IR light output voltage and a red light output voltage in a case where there is a change in a blood oxygen concentration;

FIG. 30B is a scatter diagram illustrating an example of a correlation between an IR light output voltage and a red light output voltage in a case where there is no change in a blood oxygen concentration;

FIG. 31 is a flowchart illustrating an example of a process flow of the biological information measurement program according to the third exemplary embodiment;

FIG. 32 is a graph illustrating an example of an amplitude of an IR light signal and an amplitude of a red light signal according to the third exemplary embodiment;

FIG. 33A, FIG. 33B, and FIG. 33C are graphs illustrating examples of a relationship between a coefficient and a pulse wave difference according to the third exemplary embodiment;

FIG. 34 is a graph illustrating an example of time series data of the IR light signal and time series data of the red light signal according to the third exemplary embodiment;

FIG. 35 is a graph illustrating an example of the time series data of the IR light signal and the time series data of the red light signal after correction according to the third exemplary embodiment;

FIG. 36 is a graph illustrating an example of a monitor result by the pulse wave difference according to the third exemplary embodiment;

FIG. 37 is a graph illustrating an example of the LFCT specified on the basis of the pulse wave difference according to the third exemplary embodiment;

FIG. 38A is a graph illustrating time-series data of the IR light signal and the red light signal related to a first inappropriate pattern;

FIG. 38B is a graph illustrating the first inappropriate pattern of the pulse wave difference;

FIG. 38C is a scatter diagram illustrating a correlation between the IR light output voltage and the red light output voltage with respect to the first inappropriate pattern;

FIG. 39A is a graph illustrating time-series data of the IR light signal and the red light signal related to a second inappropriate pattern;

FIG. 39B is a graph illustrating the second inappropriate pattern of the pulse wave difference;

FIG. 39C is a scatter diagram illustrating a correlation between the IR light output voltage and the red light output voltage with respect to the second inappropriate pattern;

FIG. 40A is a graph illustrating time-series data of the IR light signal and the red light signal related to a third inappropriate pattern;

FIG. 40B is a graph illustrating the third inappropriate pattern of the pulse wave difference;

FIG. 40C is a scatter diagram illustrating a correlation between the IR light output voltage and the red light output voltage with respect to the third inappropriate pattern;

FIG. 41A is a graph illustrating time-series data of the IR light signal and the red light signal related to a fourth inappropriate pattern;

FIG. 41B is a graph illustrating the fourth inappropriate pattern of the pulse wave difference;

FIG. 41C is a scatter diagram illustrating a correlation between the IR light output voltage and the red light output voltage with respect to the fourth inappropriate pattern;

FIG. 42 is a scatter diagram illustrating an example of a correlation between the IR light output voltage and the red light output voltage according to a fourth exemplary embodiment;

FIG. 43 illustrates an example of a preparation period, a breath-hold period, and an over-time observation period in an LFCT measurement;

FIG. 44A illustrates time-series data of the IR light signal and the red light signal;

FIG. 44B illustrates the LFCT of the pulse wave difference;

FIG. 45A is a graph for describing the correlation between the IR light signal and the red light signal in a region (1) in FIG. 44A;

FIG. 45B is a graph for describing the correlation between the IR light signal and the red light signal in a region (2) in FIG. 44A;

FIG. 45C is a graph for describing the correlation between the IR light signal and the red light signal in a region (3) in FIG. 44A;

FIG. 46A is a graph illustrating time-series data of the IR light signal and the red light signal during a contraction period and an expansion period;

FIG. 46B is a scatter diagram illustrating a correlation between the IR light output voltage and the red light output voltage during the contraction period and the expansion period;

FIG. 47A is a scatter diagram illustrating an example of regression lines during the contraction period and the expansion period in a case where the blood oxygen concentration does not change; and

FIG. 47B is a scatter diagram illustrating an example of regression lines during the contraction period and the expansion period in a case where the blood oxygen concentration changes.

### DETAILED DESCRIPTION

Now, exemplary embodiments for implementing the technique of the present disclosure will be described below in detail with reference to the drawings. Note that structural elements and processes having the same or substantially the same operation, effect, or function are denoted by the same or similar reference numerals throughout the drawings, and repeated description will be omitted as appropriate. Each drawing is schematically illustrated such that the technique of the present disclosure is sufficiently comprehensible. Therefore, the technique of the present disclosure is not limited to the illustrated examples. In addition, the exemplary embodiments may omit description for configurations that are not directly relevant to the present disclosure and well-known configurations.

### First Exemplary Embodiment

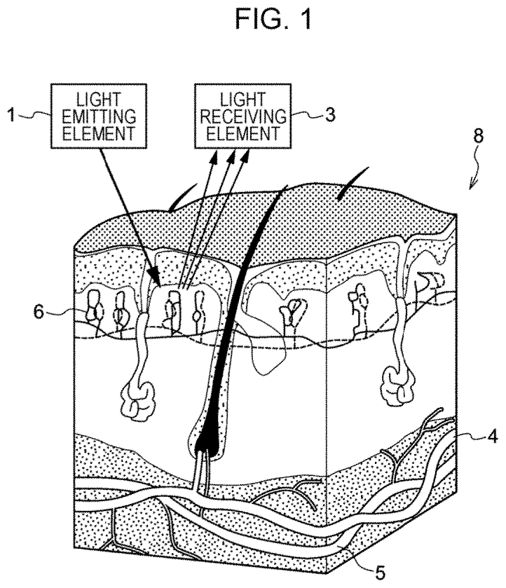

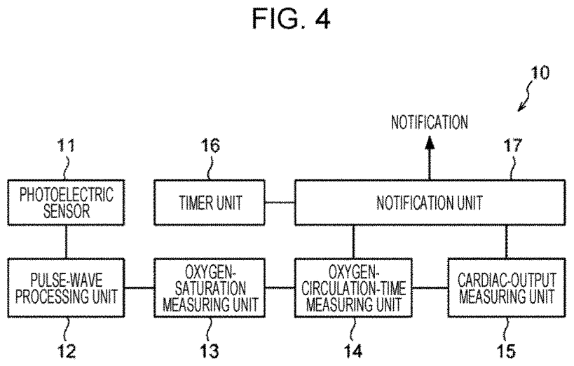

A biological information measurement apparatus 10 (FIG. 4) is an apparatus that measures information (biological information) regarding a living body 8 (FIG. 1), in particular, biological information regarding a circulatory system. The circulatory system is a general term of a group of organs for transporting body fluids such as blood while circulating them inside the body.

There are plural indicators of the biological information regarding the circulatory system. An example of an indicator indicating the state of a heart, which pumps blood through blood vessels, is cardiac output (CO) that represents the amount of blood the heart pumps out.

It is known that, when the cardiac output falls below a reference value, for example, left-sided heart failure is suspected, and when the cardiac output increases above the reference value, for example, right-sided heart failure is suspected. In this manner, the cardiac output is used in examinations for various types of heart disease and determination of medication effects.

An example of a method of measuring the cardiac output is as follows. A catheter having a balloon on the tip thereof is inserted into the pulmonary artery of a test subject whose cardiac output is to be measured, and the oxygen saturation in the blood is measured while the balloon is inflated and deflated. Then, the cardiac output is calculated from the measured oxygen saturation. The oxygen saturation in the blood herein is an example of an indicator indicating the blood oxygen concentration and is an indicator indicating how much hemoglobin in the blood is bonded to oxygen. This indicator also indicates that a symptom such as anemia is more likely to occur as the oxygen saturation in the blood decreases. The oxygen saturation in the blood is also an example of the biological information as well as the cardiac output and is used as an indicator indicating the test subject's strength for taking oxygen into the body. Hereinafter, the oxygen saturation in the blood will be simply referred to as “oxygen saturation”.

In the measurement of the oxygen saturation and cardiac output using a catheter, since a catheter needs to be inserted into a blood vessel of a test subject, a surgical procedure is necessary, and this method is more invasive to the test subject than other measurement methods.

Accordingly, in order to reduce the burden on a test subject to be less than the burden that will be imposed on the test subject in a case of employing the measurement method using a catheter, a method of measuring the oxygen saturation and cardiac output by using a pulse wave of the test subject is employed. Note that the pulse wave is an indicator indicating a pulsatile change in blood vessels in response to the heart pumping out blood.

First, a method of measuring the oxygen saturation, which is a type of biological information, will be described with reference to FIG. 1.

As illustrated in FIG. 1, a light emitting element 1 radiates light onto a test subject's body (living body 8). The light is reflected on or passes through arteries 4, veins 5, capillaries 6, and the like, which are running through the inside of the body of the test subject. The light is received by a light receiving element 3, and the intensity of the light, that is, the amount of light received from the reflected light or transmitted light, is used for measuring the oxygen saturation.

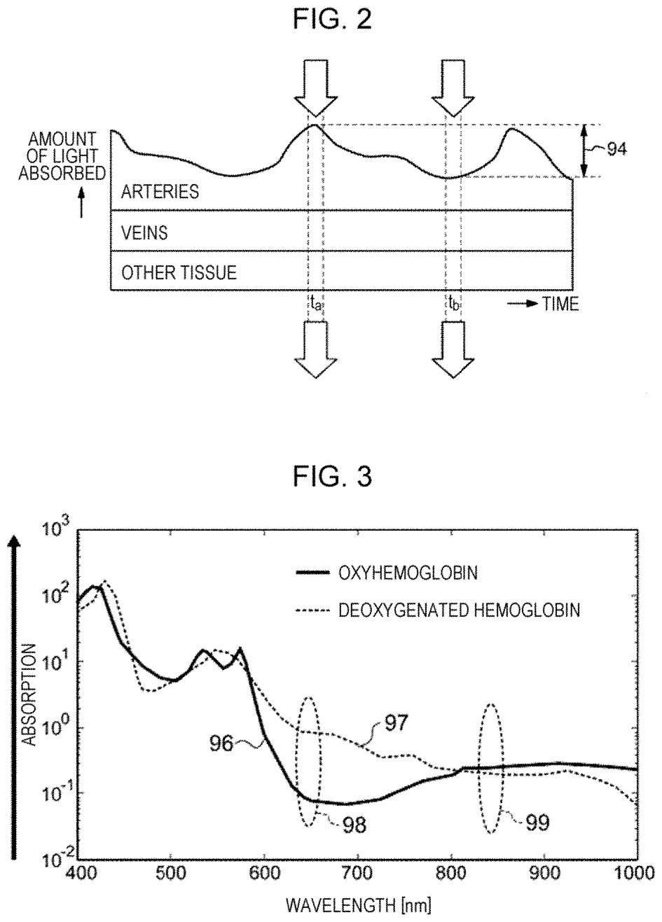

FIG. 2 is a conceptual diagram illustrating, for example, changes in the amount of light absorbed by the living body 8. As illustrated in FIG. 2, the amount of light absorbed by the living body 8 tends to vary over time.

Further examination of the variations in the amount of light absorbed by the living body 8 reveals that the amount of light absorbed by the arteries 4 varies widely, whereas for the veins 5 and other tissue including stationary tissue, the amount of variation is small enough to consider that there are no variations in the amount of light absorbed as compared with the amount of light absorbed by the arteries 4. This is because arterial blood that the heart pumps out flows through blood vessels in association with pulse waves to cause the arteries 4 to expand and contract over time in the cross-sectional direction of the arteries 4, and the thickness of the arteries 4 change. Note that the range indicated by an arrow 94 in FIG. 2 denotes the amount of variation in the amount of light absorbed corresponding to the change in the thickness of the arteries 4.

In FIG. 2, if an amount of light received at time tₐ is denoted by Iₐ and an amount of light received at time t_{b} is denoted by I_{b}, a change ΔA in the amount of light absorbed due to the change in the thickness of the arteries 4 is expressed by Formula (1).

ΔA=1n(I_{b}/Iₐ) (1)

In contrast, FIG. 3 is a graph illustrating examples of the amount of light absorbed by hemoglobin (oxyhemoglobin) bonded to oxygen flowing through the arteries 4 and the amount of light absorbed by hemoglobin (deoxygenated hemoglobin) not bonded to oxygen at different wavelengths. In FIG. 3, an absorption curve 96 represents the amount of light absorbed by oxyhemoglobin, and an absorption curve 97 represents the amount of light absorbed by deoxygenated hemoglobin.

As illustrated in FIG. 3, it is known that oxyhemoglobin is more likely than deoxygenated hemoglobin to absorb light in an infrared (IR) region 99 having a wavelength of about 850 nm to 880 nm, and that deoxygenated hemoglobin is more likely than oxyhemoglobin to absorb light, particularly light in a red region 98 having a wavelength of about 660 nm to 665 nm.

It is also known that oxygen saturation is proportional to the ratio of the change ΔA in the amount of light absorbed at different wavelengths.

Accordingly, infrared light (IR light) and red light, with which the difference between the amount of light absorbed by oxyhemoglobin and the amount of light absorbed by deoxygenated hemoglobin is more likely to become noticeable than with combinations of other wavelengths, are used to calculate the ratio between a change ΔA_{IR} in the amount of light absorbed in a case where IR light is radiated onto the living body 8 and a change ΔA_{Red} in the amount of light absorbed in a case where red light is radiated onto the living body 8. Thus, oxygen saturation S is calculated according to Formula (2). Note that k is a constant of proportionality in Formula (2).

S=k(ΔA_{Red}/ΔA_{IR}) (2)

In other words, to calculate the oxygen saturation, plural light emitting elements 1 radiate light of different wavelengths onto the living body 8. More specifically, a light emitting element 1 radiates IR light and another light emitting element 1 radiates red light onto the living body 8. In this case, a light emission period of the light emitting element 1 radiating IR light and a light emission period of the light emitting element 1 radiating red light may overlap each other. Desirably, the light emission periods do not overlap each other. Then, reflected light or transmitted light from each of the light emitting elements 1 is received by the light receiving element 3. On the basis of amounts of light received at light receiving points in time, and according to Formula (1) and Formula (2), or known formulae transformed from Formula (1) and Formula (2), the oxygen saturation is measured.

As a known formula transformed from Formula (1) above, for example, Formula (1) may be expanded to Formula (3) to express the change ΔA in the amount of light absorbed.

ΔA=1nI_{b}−1nIₐ (3)

In addition, Formula (1) may be transformed into Formula (4).

ΔA=1n(I_{b}/Iₐ)=1n(1+(I_{b}−Iₐ)/Iₐ) (4)

In general, since (I_{b}−Iₐ)<<Iₐ, 1n(I_{b}/Iₐ)≈(I_{b}−Iₐ)/Iₐ is satisfied. Accordingly, Formula (5) may be used instead of Formula (1) as the change ΔA in the amount of light absorbed.

ΔA≈(I_{b}−Iₐ)/Iₐ (5)

In the following description, when it is necessary to distinguish the light emitting element 1 that radiates IR light and the light emitting element 1 that radiates red light from each other, the light emitting element 1 that radiates IR light will be referred to as “light emitting element 1A”, and the light emitting element 1 that radiates red light will be referred to as “light emitting element 1B”.

In the above-described method, the oxygen saturation is measured by bringing the light emitting element 1 and the light receiving element 3 close to a body surface of a test subject. Thus, the burden on the test subject is less than the burden that will be imposed on the test subject in a case where the oxygen saturation is measured by inserting a catheter into a blood vessel.

The test subject's cardiac output is calculated by using the measured oxygen saturation. Details of a calculation method will be described later.

FIG. 4 is a diagram illustrating a configuration example of the biological information measurement apparatus 10. As illustrated in FIG. 4, the biological information measurement apparatus 10 includes a photoelectric sensor 11, a pulse-wave processing unit 12, an oxygen-saturation measuring unit 13, an oxygen-circulation-time measuring unit 14, a cardiac-output measuring unit 15, a timer unit 16, and a notification unit 17.

The photoelectric sensor 11 includes the light emitting element 1A that radiates IR light having a central wavelength of about 850 nm, the light emitting element 1B that radiates red light having a central wavelength of about 660 nm, and the light receiving element 3 that receives the IR light and the red light.

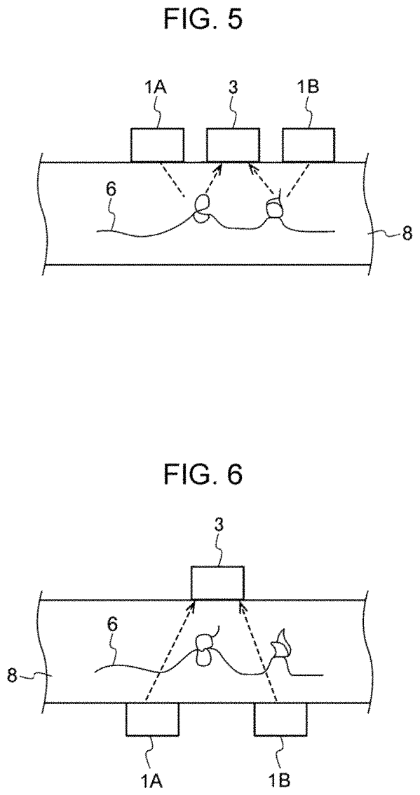

FIG. 5 illustrates an arrangement example of the light emitting element 1A, the light emitting element 1B, and the light receiving element 3 in the photoelectric sensor 11. As illustrated in FIG. 5, the light emitting element 1A, the light emitting element 1B, and the light receiving element 3 are arranged side by side in one direction on the surface of the living body 8. In this case, the light receiving element 3 receives IR light and red light reflected on the capillaries 6 and the like in the living body 8.

However, the arrangement of the light emitting element 1A, the light emitting element 1B, and the light receiving element 3 is not limited to the arrangement example illustrated in FIG. 5. For example, as illustrated in FIG. 6, the light emitting element 1A, the light emitting element 1B, and the light receiving element 3 may also be arranged such that the light emitting elements 1A and 1B face the light receiving element 3 with the living body 8 sandwiched between the light emitting elements 1A and 1B and the light receiving element 3. In this case, the light receiving element 3 receives IR light and red light transmitted through the living body 8.

As an example herein, each of the light emitting element 1A and the light emitting element 1B will be described as a surface-emitting laser element such as a vertical-cavity surface-emitting laser (VCSEL). However, each of the light emitting element 1A and the light emitting element 1B is not limited to a surface-emitting laser element and may be an edge-emitting laser element. Alternatively, each of the light emitting element 1A and the light emitting element 1B may be a light emitting diode (LED).

The photoelectric sensor 11 includes a clip (not illustrated) that is used for attaching the photoelectric sensor 11 to a body part of a test subject, and, with the clip, the photoelectric sensor 11 is attached to the test subject to be in contact with the body surface of the test subject so that IR light and red light do not leak from the photoelectric sensor 11 to the outside. In order for the light receiving element 3 to receive, as accurately as possible, IR light and red light reflected on or transmitted through the living body 8 of the test subject, the photoelectric sensor 11 may be positioned so as to be in contact with the body surface of the test subject. However, the photoelectric sensor 11 may also be disposed at a position away from the body surface but within an area in which the light receiving element 3 receives IR light and red light reflected on or transmitted through the living body 8 of the test subject.

The photoelectric sensor 11 converts the amounts of IR light and red light received by the light receiving element 3 into, for example, voltage values and informs the pulse-wave processing unit 12 of the voltage values.

The light emitting element 1A and the light emitting element 1B each radiate a predetermined amount of light, and thus, from the amounts of IR light and red light received by the photoelectric sensor 11, the amounts of IR light and red light absorbed by the living body 8 are obtained.

Thus, the pulse-wave processing unit 12 generates, by using the amounts of IR light and red light received from the photoelectric sensor 11, a pulse-wave signal indicating the pulse wave of the test subject obtained from the IR light and a pulse-wave signal indicating the pulse wave of the test subject obtained from the red light. The pulse-wave processing unit 12 amplifies the voltage values corresponding to the amounts of IR light and red light received, within a predetermined range suitable for generating the pulse-wave signals. Then, the pulse-wave processing unit 12 generates the pulse-wave signals from which a noise component is removed by using a known filter or the like. The pulse-wave processing unit 12 informs the oxygen-saturation measuring unit 13 of the generated pulse-wave signals.

Upon receipt of the pulse-wave signals from the pulse-wave processing unit 12, the oxygen-saturation measuring unit 13 measures the oxygen saturation of the test subject by using the received pulse-wave signals. More specifically, on the basis of the pulse-wave signals and according to Formula (1), the oxygen-saturation measuring unit 13 calculates the change ΔA_{IR} in the amount of IR light absorbed and the change ΔA_{Red} in the amount of red light absorbed due to the change in the thickness of the arteries 4. Furthermore, by using the calculated change ΔA_{IR} and change ΔA_{Red} and, for example, according to Formula (2), the oxygen-saturation measuring unit 13 measures the oxygen saturation of the test subject and informs the oxygen-circulation-time measuring unit 14 of the measured oxygen saturation.

As an example, a case where the oxygen-saturation measuring unit 13 measures the oxygen saturation of a test subject will be described below. However, the oxygen-saturation measuring unit 13 may measure any value as long as the value indicates a temporal change in the oxygen saturation of the test subject. For example, the oxygen-saturation measuring unit 13 may measure a value that correlates with the temporal change in the oxygen saturation, such as a reciprocal of the oxygen saturation or the ratio between the change ΔA_{Red} and the change ΔA_{IR}.

By referring to the oxygen saturation of the test subject measured by the oxygen-saturation measuring unit 13, the oxygen-circulation-time measuring unit 14 detects an inflection point of the oxygen saturation and measures the oxygen circulation time.

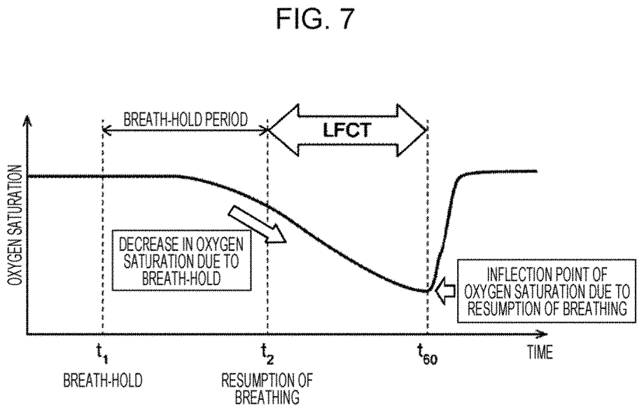

The graph in FIG. 7 illustrates an example of changes in the oxygen saturation at a specific body part of a test subject. In the graph, the horizontal axis represents time, while the vertical axis represents the oxygen saturation.

When the test subject holds their breath at time t₁, the oxygen saturation in the test subject starts to decrease. Even if the test subject resumes breathing upon an end of a breath-hold period during which the test subject holds their breath (time t₂), it takes time for oxygen taken into blood by resumption of breathing to reach the specific body part from the lungs, and thus, the oxygen saturation in the test subject keeps decreasing after time t₂. The oxygen taken in blood by resumption of breathing eventually reaches the specific body part from the lungs, and thus, the oxygen saturation in the test subject starts to increase. The point at which the oxygen saturation turns from a decrease to an increase will be referred to as “inflection point”. If the time at which the inflection point appears is referred to as time t₆₀, the oxygen circulation time is denoted by the difference between time t₂ and time t₆₀.

In other words, the oxygen circulation time corresponds to the time taken for oxygen to be transported from the lungs to the specific body part and is also called “oxygen transport time”.

The measurement accuracy of the oxygen circulation time measured by using the change in the oxygen saturation tends to differ due to differences in the breath-hold period. Thus, in the biological information measurement apparatus 10, a breath-hold time T1 that defines the length of the breath-hold period is set in advance.

More specifically, if the test subject resumes breathing while the oxygen saturation keeps decreasing, the oxygen saturation may increase before decreasing to a lowest value, the lowest value being necessary to measure the oxygen circulation time. If the oxygen circulation time is measured by using a change in the oxygen saturation that has not decreased to the lowest value necessary to measure the oxygen circulation time, the measurement accuracy of the oxygen circulation time is lower than that of the oxygen circulation time measured by using a change in the oxygen saturation that has decreased to the lowest value necessary to measure the oxygen circulation time.

Therefore, the breath-hold time T1 is set such that a lowest value of the oxygen saturation obtained by as many test subjects as possible holding their breath falls below the lowest value of the oxygen saturation necessary to measure the oxygen circulation time.

Thus, the breath-hold time T1 is prescribed in advance by experiment or the like using an actual machine using the biological information measurement apparatus 10.

In the following description, the lowest value of the oxygen saturation necessary to measure the oxygen circulation time will be referred to as “reference value H of the oxygen saturation”. The reference value H of the oxygen saturation is also prescribed in advance by experiment or the like using an actual machine using the biological information measurement apparatus 10.

Note that the lowest value of the oxygen saturation falling below the reference value H of the oxygen saturation reference means the lowest value of the oxygen saturation becoming less than or equal to the reference value H of the oxygen saturation.

The oxygen-circulation-time measuring unit 14 stores, as time t₆₀, the time at which the inflection point of the oxygen saturation is detected, and measures the time represented by the difference between time t₂ and time t₆₀ as the oxygen circulation time. To “detect the inflection point” includes a case of detecting a position before or after the inflection point of the oxygen saturation on the time axis within a range that does not substantially affect the measurement of the oxygen circulation time.

The oxygen-circulation-time measuring unit 14 informs the notification unit 17 and the cardiac-output measuring unit 15 of the oxygen circulation time.

Note that the body part at which the oxygen circulation time is measured is determined by a position on the test subject to which the photoelectric sensor 11 is attached. In the present exemplary embodiment, as an example, the photoelectric sensor 11 is attached to a peripheral body part of the test subject. More specifically, the photoelectric sensor 11 is attached to a fingertip, and the oxygen circulation time in a case where oxygen is transported from the lungs to the fingertip is measured. This is because the fingertip is farther from the lungs than other body parts are, and accordingly, the oxygen circulation time is longer, so that the oxygen circulation time may be obtained with an accuracy higher than that in a case where the photoelectric sensor 11 is attached to one of the other body parts. Note that the term “peripheral body part” refers to a body part that is further toward the distal side than the neck, the shoulders, and the hip joint are in the body of the test subject.

Thus, the oxygen circulation time from the lungs to the fingertip may also be particularly referred to as “lung-to-finger circulation time (LFCT)”. The present exemplary embodiment will describe an example in which the photoelectric sensor 11 is attached to a fingertip of a test subject and the LFCT is measured by the oxygen-circulation-time measuring unit 14. However, the body part to which the photoelectric sensor 11 is attached is not limited to a fingertip. The photoelectric sensor 11 may be attached to any body part of a test subject as long as errors in the measurement of the oxygen circulation time are within a predetermined range. Note that, although the term “fingertip” refers to a fingertip or a thumb tip of a test subject, the photoelectric sensor 11 may be attached to a tip of a toe of the test subject.

The cardiac-output measuring unit 15 measures the cardiac output of the test subject by using the LFCT received from the oxygen-circulation-time measuring unit 14. The cardiac output is calculated by using, for example, a calculation formula that is obtained beforehand and that represents the relationship between the LFCT and the cardiac output.

Note that the cardiac-output measuring unit 15 may measure information related to the cardiac output in addition to the cardiac output. The term “information related to the cardiac output” refers to information that is considered to be correlated with the cardiac output and includes, for example, a cardiac index and a stroke volume.

The term “cardiac index” is a value that is obtained by dividing the cardiac output of a test subject by the body surface area of the test subject so as to accommodate variations in cardiac output between test subjects due to physical differences. The term “stroke volume” is a value that indicates the amount of blood that the heart pumps out to the arteries 4 in a single contraction and is obtained by dividing the cardiac output by the number of heartbeats of the test subject per minute.

The cardiac-output measuring unit 15 informs the notification unit 17 of the measured cardiac output. Note that the biological information measurement apparatus 10 illustrated in FIG. 4 includes the cardiac-output measuring unit 15 for measuring the cardiac output, but the biological information measurement apparatus 10 does not necessarily measure the cardiac output. Thus, the cardiac-output measuring unit 15 may be absent in the biological information measurement apparatus 10.

The notification unit 17 gives a notification of a breath-hold instruction to the test subject. The notification unit 17 also measures the breath-hold time T1 in cooperation with the timer unit 16, and, after the breath-hold time T1 has elapsed, gives a notification of a breathing-resumption instruction to the test subject. Note that the timer unit 16 includes a timer for measuring time, and measures the time of a segment for which a start and an end are designated.

To measure the oxygen circulation time of the same test subject plural times, the notification unit 17 gives a notification of a breathing-resumption instruction to the test subject and then gives a notification of a breath-hold instruction again to the test subject for a subsequent measurement of the oxygen circulation time.

The period from when the test subject who is instructed to resume breathing after a breath-hold until the test subject is instructed to hold their breath again for the subsequent measurement of the oxygen circulation time is a period during which the test subject adjusts their breath before the subsequent measurement of the oxygen circulation time. Thus, in the following description, the length of the period will be referred to as “breath-adjusting time T2”.

Furthermore, the notification unit 17 gives a notification of the oxygen circulation time measured by the oxygen-circulation-time measuring unit 14 and the cardiac output measured by the cardiac-output measuring unit 15 to at least one of the test subject and a medical worker who is in charge of the test subject.

The term “notification” in the present exemplary embodiment means to enable at least one of the test subject and a medical worker who is in charge of the test subject to notice an instruction from the biological information measurement apparatus 10 or information obtained by the biological information measurement apparatus 10. Thus, to give a notification of an instruction to resume breathing or hold their breath to the test subject by using the biological information measurement apparatus 10 includes: visual notification, such as displaying the instruction on a display; auditory notification, such as outputting the instruction as speech; and tactile notification for the test subject, such as transmitting the instruction by vibration. In addition, to give a notification of biological information such as the oxygen circulation time and the cardiac output measured by the biological information measurement apparatus 10 includes: visual notification, such as displaying the measured biological information on a display; auditory notification, such as outputting the measured biological information as speech; notification using a storage device, such as storing the measured biological information on a storage device that at least one of the test subject and a medical worker who is in charge of the test subject is authorized to read; notification using a recording medium, such as forming the measured biological information on a recording medium, such as a sheet, by using an image forming apparatus; and notification using a communication line, such as transmitting the measured biological information to an external apparatus via a communication line.

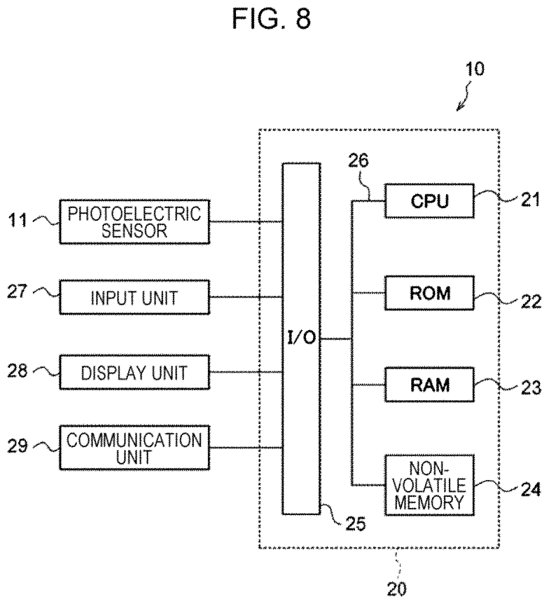

The above-described biological information measurement apparatus 10 is constituted by, for example, a computer. FIG. 8 is a diagram illustrating a configuration example of a principal portion in an electrical system of the biological information measurement apparatus 10 constituted by a computer 20.

The computer 20 includes a central processing unit (CPU) 21, a read only memory (ROM) 22, a random access memory (RAM) 23, a non-volatile memory 24, and an input/output (I/O) interface 25. The CPU 21 performs processes of the pulse-wave processing unit 12, the oxygen-saturation measuring unit 13, the oxygen-circulation-time measuring unit 14, the cardiac-output measuring unit 15, the timer unit 16, and the notification unit 17 according to the present exemplary embodiment. The CPU 21, the ROM 22, the RAM 23, the non-volatile memory 24, and the I/O interface 25 are connected to one another via a bus 26. Note that there is no limitation on an operating system used by the computer 20.

The non-volatile memory 24 is an example of a storage device that maintains information stored therein even when supply of power to the non-volatile memory 24 is discontinued, and for example, a semiconductor memory is used. However, the non-volatile memory 24 may also be a hard disk.

For example, the photoelectric sensor 11, an input unit 27, a display unit 28, and a communication unit 29 are connected to the I/O interface 25.

The photoelectric sensor 11 and the I/O interface 25 are connected to each other in a wired or wireless manner. Note that the biological information measurement apparatus 10 and the photoelectric sensor 11 may be provided as separate units so as to be separated from each other, or the biological information measurement apparatus 10 and the photoelectric sensor 11 may be accommodated in the same housing so as to be integrated with each other.

The input unit 27 is, for example, an input device that receives an instruction from a user of the biological information measurement apparatus 10 and informs the CPU 21 of the instruction. The input unit 27 includes, for example, a button, a touch panel, a keyboard, a mouse, and the like. The user of the biological information measurement apparatus 10 includes, for example, a test subject and a medical worker who is in charge of the test subject.

The display unit 28 is a display device that displays, for example, information processed by the CPU 21 to the user of the biological information measurement apparatus 10. As the display unit 28, for example, a display device such as a liquid crystal display, an organic electroluminescent (EL) display, or a projector is used.

Note that the biological information measurement apparatus 10 does not necessarily include the display unit 28, and a unit corresponding to the notification manner of instructions for the user and biological information is connected to the I/O interface 25.

For example, in a case where a notification of an instruction from the biological information measurement apparatus 10 and measured biological information is to be given as speech to the user of the biological information measurement apparatus 10, a speaker unit may be connected to the I/O interface 25. In addition, in a case where a notification of an instruction from the biological information measurement apparatus 10 is to be given tactilely to the user of the biological information measurement apparatus 10, a vibration unit may be connected to the I/O interface 25.

The communication unit 29 includes a communication protocol that is used for connecting, for example, a communication line such as the Internet and the biological information measurement apparatus 10 to each other and performs data communication between the biological information measurement apparatus 10 and other external apparatuses connected to the communication line. The connection form to the communication line in the communication unit 29 may be wired or wireless. If data communication between the biological information measurement apparatus 10 and other external apparatuses connected to the communication line is unnecessary, it is unnecessary to connect the communication unit 29 to the I/O interface 25.

A unit to be connected to the I/O interface 25 is not limited to those examples described above, and for example, another unit such as a printing unit that prints measured biological information on a recording medium may be connected to the I/O interface 25.

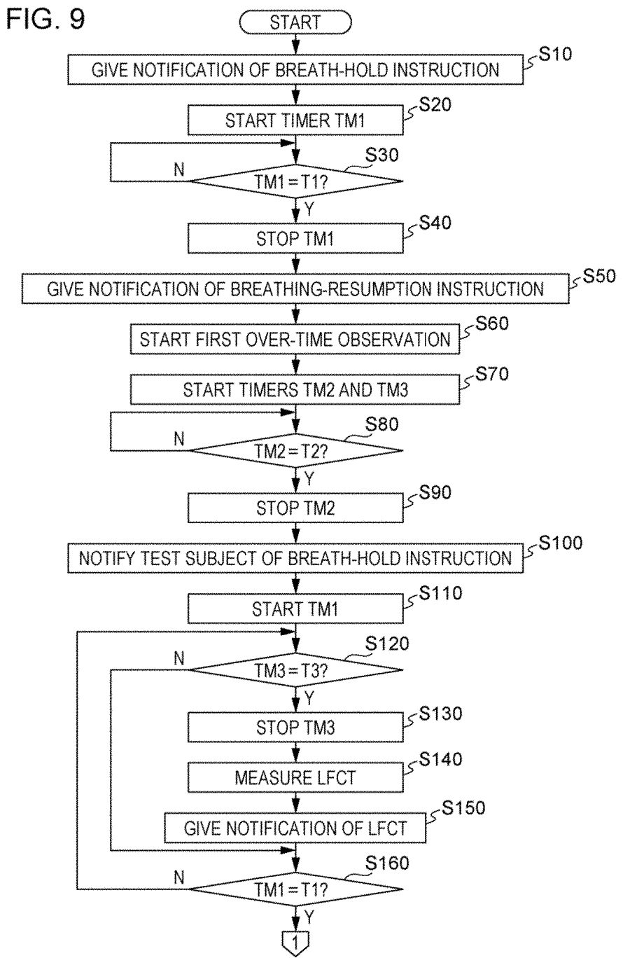

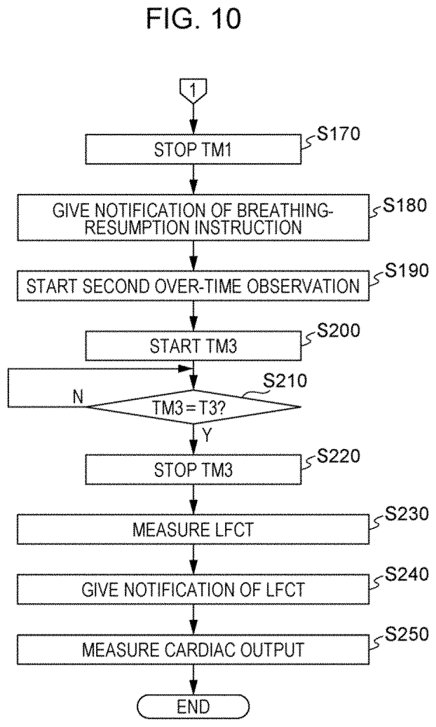

Now, operations of the biological information measurement apparatus 10 will be described below with reference to FIG. 9 and FIG. 10. The following description illustrates an example in which, upon receipt of an instruction for measurement start from a user through the input unit 27, the biological information measurement apparatus 10 measures the LFCT, which is an example of the oxygen circulation time, plural times. The number of times the LFCT is to be measured is stored in advance in the non-volatile memory 24 of the biological information measurement apparatus 10. The number of times the LFCT is to be measured, stored in the non-volatile memory 24, is changeable by the user. In response to a single measurement start instruction, the number of times the LFCT is to be measured by the biological information measurement apparatus 10 may be any number of two or more. Note that the user may designate the number of times the LFCT is to be measured, when issuing a measurement start instruction.

For the convenience of description, “twice” is set as the number of times the LFCT is to be measured, in the non-volatile memory 24 of the biological information measurement apparatus 10. That is, the biological information measurement apparatus 10 successively measures the LFCT twice in response to a single measurement start instruction.

In addition, as described above with reference to FIG. 7, the time, from when a test subject resumes breathing after a breath-hold until the inflection point of the oxygen saturation appears, differs for each test subject. Thus, a reference time for prescribing how long the measurement of the oxygen saturation is continued from when a test subject resumes breathing is set in the biological information measurement apparatus 10. This reference time is referred to as “over-time observation time T3”.

For example, the over-time observation time T3 is set to be longer than an average LFCT so that the LFCT can be measured for a test subject for whom the inflection point of the oxygen saturation appears later than that of other test subjects. More specifically, the over-time observation time T3 is prescribed in advance by experiment or the like using an actual machine using the biological information measurement apparatus 10. The observation time T3 is an example of “oxygen-circulation-time measurement period” according to the present exemplary embodiment.

In addition to the over-time observation time T3, the breath-hold time T1 and the breath-adjusting time T2, each of which is determined in advance, are stored in the non-volatile memory 24 of the biological information measurement apparatus 10.

As an example, the breath-hold time T1, the breath-adjusting time T2, and the over-time observation time T3 have the following relationship.

Relationship 1: breath-adjusting time T2<over-time observation time T3

Relationship 2: (breath-hold time T1+breath-adjusting time T2)>over-time observation time T3

FIG. 9 and FIG. 10 are a flowchart illustrating an example of a flow of a biological information measurement process executed by the CPU 21 upon receipt of a measurement start instruction from a user through the input unit 27 in a state where the photoelectric sensor 11 is attached to a fingertip of a test subject.

A biological information measurement program that prescribes the biological information measurement process is, for example, stored in advance in the ROM 22 of the biological information measurement apparatus 10. The CPU 21 of the biological information measurement apparatus 10 reads the biological information measurement program stored in the ROM 22 and executes the biological information measurement process.

Upon receipt of a measurement start instruction, the biological information measurement apparatus 10 starts measuring oxygen saturation and stores the measured oxygen saturation in, for example, the RAM 23.

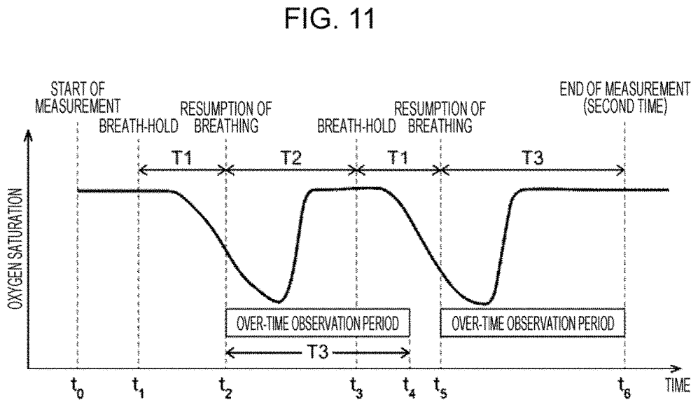

Note that FIG. 11 is a graph illustrating a temporal change example of the oxygen saturation of a test subject obtained through the biological information measurement process. Now, the flow of the biological information measurement process will be described below with reference to the graph in FIG. 11. In the graph in FIG. 11, the biological information measurement apparatus 10 receives a measurement start instruction from the user through the input unit 27 at time t₀.

First, in step S10 in FIG. 9, the CPU 21 gives a notification of a breath-hold instruction to a test subject as a preparation for a first LFCT measurement. In the graph in FIG. 11, the CPU 21 gives a notification of a breath-hold instruction to the test subject at time t₁. That is, the first LFCT measurement starts at time t₁.

Since the test subject has given the notification of the breath-hold instruction, in step S20, the CPU 21 starts a timer TM1. The timer TM1 is a timer for measuring the breath-hold time T1. The CPU 21 starts the timer TM1, for example, by using a timer function incorporated in the CPU 21. If a timer function is not incorporated in the CPU 21, for example, the CPU 21 may start the timer TM1 by using a timer unit (omitted from illustration) connected to the I/O interface 25.

In step S30, the CPU 21 determines whether the timer TM1 reaches the breath-hold time T1. If the timer TM1 does not reach the breath-hold time T1, the CPU 21 repeatedly executes the determination processing in step S30 until the timer TM1 reaches the breath-hold time T1. Since the test subject holds their breath until the timer TM1 reaches the breath-hold time T1, as illustrated in the graph in FIG. 11, the oxygen saturation of the test subject falls at and after time t₁.

Note that there is a distance from the lungs to fingertips. Thus, it takes time for the photoelectric sensor 11 attached to a fingertip to detect a decrease in the oxygen saturation due to a breath-hold of the test subject. Thus, the oxygen saturation immediately after time t1 is the same as that before the breath-hold of the test subject, but then falls toward time t₂.

If it is determined in the determination processing in step S30 in FIG. 9 that the timer TM1 reaches the breath-hold time T1, the process advances to step S40.

In step S40, the CPU 21 stops the timer TM1. To “stop” the timer TM1 means to cancel the measurement of time by using the timer TM1 and set the value of the timer TM1 to “0”. In other words, to stop the timer TM1 is equivalent to reset of the timer TM1.

In step S50, the CPU 21 gives a notification of a breathing-resumption instruction to the test subject. In the graph in FIG. 11, at time t₂ at which the breath-hold time T1 has elapsed from time t₁, the CPU 21 gives a notification of a breathing-resumption instruction to the test subject. Thus, the test subject resumes breathing after a breath-hold. That is, the test subject holds their breath during the breath-hold time T1.

As described above, since there is a distance from the lungs to fingertips, it takes time for the photoelectric sensor 11 attached to a fingertip to detect an increase in the oxygen saturation due to resumption of breathing of the test subject even if the test subject resumes breathing at time t₂. Thus, the oxygen saturation falls after time t2.

Since the test subject resumes breathing, in step S60, the CPU 21 starts a first oxygen-saturation over-time observation. To “start an oxygen-saturation over-time observation” means a state where the CPU 21 associates the time (in this case, time t₂) at which the test subject resumes breathing and the oxygen saturation measured at time t₂ at which the test subject resumes breathing with each other and detects a change in the oxygen saturation to be used for measuring the LFCT.

Thus, in step S70, the CPU 21 starts a timer TM2 and a timer TM3. The timer TM2 is a timer for measuring the breath-adjusting time T2. The timer TM3 is a timer for measuring the over-time observation time T3. That is, until the over-time observation time T3 elapses from time t₂, the CPU 21 continues the measurement of the oxygen saturation.

On the other hand, the test subject who has resumed breathing adjusts breathing for a second LFCT measurement.

In step S80, the CPU 21 determines whether the timer TM2 that is started in step S70 reaches the breath-adjusting time T2. If the timer TM2 does not reach the breath-adjusting time T2, the CPU 21 repeatedly performs the determination processing in step S80 until the timer TM2 reaches the breath-adjusting time T2.

If it is determined in the determination processing in step S80 that the timer TM2 reaches the breath-adjusting time T2, the process advances to step S90. In step S90, the CPU 21 stops the timer TM2.

In step S100, the CPU 21 gives a notification of a breath-hold instruction to the test subject as a preparation for a second LFCT measurement. In the graph in FIG. 11, the CPU 21 gives a notification of a breath-hold instruction to the test subject at time t₃. That is, the second LFCT measurement starts at time t₃.

Since the test subject is given a notification of a breath-hold instruction, in step S110, the CPU 21 starts the timer TM1.

As described above, because of the relationship “breath-adjusting time T2<over-time observation time T3”, the first LFCT measurement is continued at and after time t₃. That is, at and after time t₃, at which the test subject holds their breath for the second LFCT measurement, the CPU 21 executes the first LFCT measurement concurrently. Thus, the LFCT of the test subject can be measured in such a manner that the inflection point of the oxygen saturation appears after an elapse of the breath-adjusting time T2 even when the second LFCT measurement starts.

In step S120, the CPU 21 determines whether the timer TM3 started in step S70 reaches the over-time observation time T3. If the timer TM3 does not reach the over-time observation time T3, the process advances to step S160 to continue the first LFCT measurement.

In step S160, the CPU 21 determines whether the timer TM1 started in step S110 reaches the breath-hold time T1. If the timer TM1 does not reach the breath-hold time T1, the process advances to step S120. That is, the CPU 21 alternately executes the determination processing in step S120 and the determination processing in step S160 until the timer TM3 reaches the over-time observation time T3.

If it is determined in the determination processing in step S120 that the timer TM3 reaches the over-time observation time T3, the process advances to step S130. In the graph in FIG. 11, the timer TM3 reaches the over-time observation time T3 at time t₄.

Since the first oxygen-saturation over-time observation ends at time t₄, the first LFCT measurement also ends. Thus, in step S130, the CPU 21 stops the timer TM3.

In step S140, the CPU 21 obtains, from the RAM 23, time-series data of the oxygen saturation measured during the over-time observation time T3, and detects the inflection point of the oxygen saturation. Furthermore, the CPU 21 measures the period from time t₂ at which the test subject resumes breathing until the time at which the inflection point of the oxygen saturation appears, and obtains the measured period as a result of the first LFCT measurement.

In step S150, the CPU 21 gives a notification of the result of the first LFCT measurement measured in step S140 to the user, for example, by displaying it on the display unit 28. Subsequently, the CPU 21 advances to step S160 and determines whether the timer TM1 reaches the breath-hold time T1.

If it is determined in the determination processing in step S160 that the timer TM1 reaches the breath-hold time T1, the process advances to step S170 in FIG. 10.

Since the measurement of the breath-hold time T1 ends, in step S170, the CPU 21 stops the timer TM1.

In step S180, the CPU 21 gives a notification of a breathing-resumption instruction to the test subject. In the graph in FIG. 11, at time t₅, at which the breath-hold time T1 has elapsed after time t₃, the CPU 21 gives a notification of a breathing-resumption instruction to the test subject. Thus, the test subject resumes breathing after a breath-hold.

Since the test subject resumes breathing, in step S190, the CPU 21 starts a second oxygen-saturation over-time observation. Thus, in step S200, the CPU 21 starts the timer TM3. Note that in the first oxygen-saturation over-time observation, the CPU 21 starts the timer TM2 in addition to the timer TM3 in step S70 in FIG. 9. However, since the second oxygen-saturation over-time observation is the final oxygen-saturation over-time observation, the test subject does not need to adjust their breath for a subsequent LFCT measurement. Thus, the CPU 21 does not start the timer TM2.

In step S210, the CPU 21 determines whether the timer TM3 started in step S200 reaches the over-time observation time T3. If the timer TM3 does not reach the over-time observation time T3, the determination processing in step S210 is repeatedly executed.

On the other hand, if it is determined in the determination processing in step S210 that the timer TM3 reaches the over-time observation time T3, the process advances to step S220. In the graph in FIG. 11, the timer TM3 reaches the over-time observation time T3 at time t₆.

Since the second oxygen-saturation over-time observation ends at time t₆, the second LFCT measurement also ends. Thus, in step S220, the CPU 21 stops the timer TM3.

In step S230, the CPU 21 obtains, from the RAM 23, time-series data of the oxygen saturation measured during the over-time observation time T3 from time t₅ until time t₆, and detects the inflection point of the oxygen saturation. Furthermore, the CPU 21 measures the period from time t₅ at which the test subject resumes breathing until the time at which the inflection point of the oxygen saturation appears, and obtains the measured period as a result of the second LFCT measurement.

In step S240, the CPU 21 gives a notification of the result of the second LFCT measurement measured in step S230 to the user, for example, by displaying it on the display unit 28.

In step S250, the CPU 21 substitutes the first LFCT obtained in step S140 and the second LFCT measured in step S230 into, for example, the calculation formula that represents the relationship between the LFCT and the cardiac output and is obtained in advance to measure the cardiac output at each measurement.

The CPU 21 does not need to measure the cardiac output of the test subject together even if measuring the LFCT of the test subject. Thus, the CPU 21 may skip step S250.

In the above manner, the CPU 21 ends the biological information measurement process illustrated in FIG. 9 and FIG. 10.

Thus, in a case where the LFCT of the test subject is to be measured plural times in response to a single measurement start instruction, before the over-time observation time T3 of the oxygen saturation in the previous measurement ends, as a preparation for the subsequent LFCT measurement, the biological information measurement apparatus 10 gives a notification of a breath-hold instruction to the test subject.

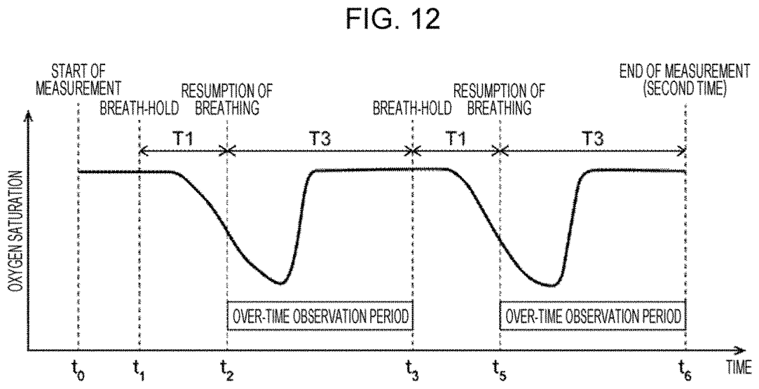

On the other hand, FIG. 12 is a graph illustrating a temporal change example of the oxygen saturation of the test subject in a case where, after the over-time observation time T3 of the oxygen saturation in the first LFCT measurement ends, the test subject is given a notification of a breath-hold instruction as a preparation for a second LFCT measurement.

In the graph illustrated in FIG. 12, the over-time observation time T3 in the first measurement from time t₂ until time t₃ and the breath-hold time T1 in the second measurement from time t₃ until time t₅ do not overlap each other. Thus, time t₆ corresponding to the end of the second LFCT measurement illustrated in FIG. 12 is later than time t₆ corresponding to the end of the second LFCT measurement illustrated in FIG. 11 by the time (t₄-t₃) in FIG. 11. That is, the biological information measurement apparatus 10 according to the present exemplary embodiment may shorten the time for measuring the LFCT plural times as compared with a case where the subsequent LFCT measurement starts after the previous LFCT measurement has ended. In addition, since the time for measuring the LFCT is shortened, the time for measuring biological information calculated from the LFCT, such as the cardiac output, is also shortened.

Note that in the biological information measurement process illustrated in FIG. 9 and FIG. 10, an example of giving a notification of the LFCT at each measurement of the LFCT has been described. However, a notification of the LFCTs measured in the respective measurements may also be given at once after a prescribed number of LFCT measurements have ended. More specifically, instead of giving a notification of the LFCT in the first measurement in step S150 in FIG. 9, a notification of the LFCT in the first measurement and the LFCT in the second measurement may be given at once in step S240 in FIG. 10.

The biological information measurement apparatus 10 does not necessarily measure the LFCT each time the LFCT is supposed to be measured. More specifically, the biological information measurement apparatus 10 does not necessarily perform steps S140, S150, S230, S240, and S250 in the biological information measurement process illustrated in FIG. 9 and FIG. 10 and may store only the change in the time-series data of the oxygen saturation in the non-volatile memory 24. Furthermore, after the biological information measurement process has been ended, the biological information measurement apparatus 10 may obtain the oxygen saturation from the non-volatile memory 24 in response to an instruction from a user and may measure the LFCT at each measurement. By storing the time-series data of the oxygen saturation in the non-volatile memory 24 after the measurement has been started, the biological information measurement apparatus 10 may obtain the measured oxygen saturation at any time afterwards, and thus, the LFCT may be measured at each measurement at a timing according to an instruction from a user. Such a method of measuring the LFCT is applied to, for example, a case where a test subject measures the oxygen saturation at home and a case where a medical worker checks the oxygen saturation and the LFCT of the test subject at hospital afterwards.

### Second Exemplary Embodiment

In the first exemplary embodiment, the over-time observation time T3 is a fixed value. However, to measure the LFCT of a test subject, the oxygen saturation may be measured from when the test subject resumes breathing until the inflection point of the oxygen saturation is detected.

Thus, the second exemplary embodiment will describe the biological information measurement apparatus 10 that performs control such that the over-time observation time T3 is changed depending on a detection status of the inflection point of the oxygen saturation in a final LFCT measurement.

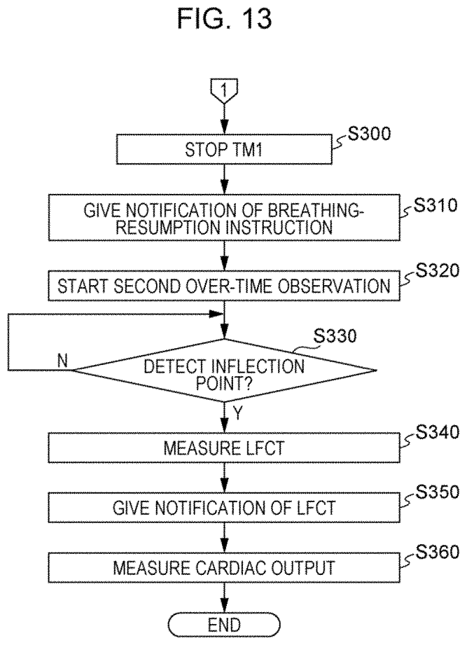

FIG. 13 is a flowchart illustrating an example of a flow of a biological information measurement process executed by the CPU 21 subsequently to the biological information measurement process according to the first exemplary embodiment illustrated in FIG. 9.

Upon receipt of a measurement start instruction, the biological information measurement apparatus 10 starts measuring oxygen saturation and stores the measured oxygen saturation, for example, in the RAM 23.

Note that the biological information measurement apparatus 10 according to the second exemplary embodiment also successively measures the LFCT twice in response to a single measurement start instruction as in the biological information measurement apparatus 10 according to the first exemplary embodiment.

In step S160 in the biological information measurement process illustrated in FIG. 9, if it is determined that the timer TM1, which has been started in response to the breath-hold of the test subject for a second time, reaches the breath-hold time T1, the process advances to step S300 in FIG. 13.

Since the second measurement of the breath-hold time T1 has ended, in step S300, the CPU 21 stops the timer TM1.

In step S310, the CPU 21 gives a notification of a breathing-resumption instruction to the test subject. Thus, the test subject resumes breathing after a breath-hold.

In response to the test subject resuming breathing, in step S320, the CPU 21 starts an oxygen-saturation over-time observation in the second, that is, final LFCT measurement.

In step S330, the CPU 21 obtains a change in the oxygen saturation during the over-time observation and determines whether the inflection point of the oxygen saturation for the test subject has been detected. If the inflection point of the oxygen saturation has not been detected, the CPU 21 repeatedly performs the determination processing in step S330 until the inflection point of the oxygen saturation is detected. On the other hand, if the inflection point of the oxygen saturation has been detected, the process advances to step S340.

In step S340, the CPU 21 measures the period from time t₅ at which the test subject resumes breathing until time t₆₀ at which the inflection point of the oxygen saturation appears, and obtains the measured period as a result of the second LFCT measurement.

In step S350, the CPU 21 gives a notification of the result of the second LFCT measurement measured in step S340 to the user, for example, by displaying it on the display unit 28.

In step S360, the CPU 21 substitutes the first LFCT obtained in step S140 in FIG. 9 and the second LFCT measured in step S340 into, for example, the calculation formula that represents the relationship between the LFCT and the cardiac output and is obtained in advance to measure the cardiac output at each measurement.

In the above manner, the CPU 21 ends the biological information measurement process according to the second exemplary embodiment illustrated in FIG. 9 and FIG. 13.

That is, the CPU 21 of the biological information measurement apparatus 10 according to the second exemplary embodiment performs control such that the period from when the test subject resumes breathing until the inflection point of the oxygen saturation is detected during the final LFCT measurement is the over-time observation time T3.

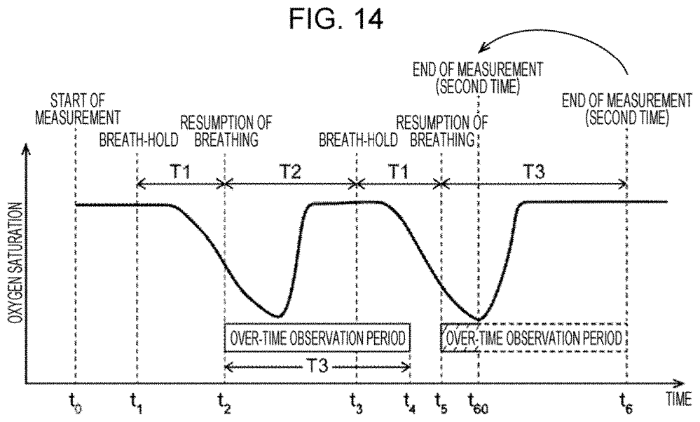

FIG. 14 is a graph illustrating a temporal change example of the oxygen saturation of the test subject in a case where the inflection point of the oxygen saturation of the test subject is detected before the predetermined over-time observation time T3 during the final LFCT measurement elapses from time t₅ at which the test subject resumes breathing, that is, unchanged over-time observation time T3 during the final LFCT measurement elapses from time t₅ at which the test subject resumes breathing.

As illustrated in FIG. 14, in a case where time t₆₀, at which the inflection point of the oxygen saturation appears is before time t₆, at which the unchanged over-time observation time T3 elapses from time t₅, the over-time observation time T3 is shortened to the period from time t₅ until time t₆₀. In the example illustrated in FIG. 14, the shortened over-time observation time T3 is hatched.

That is, the biological information measurement apparatus 10 according to the second exemplary embodiment may shorten the time that takes from the reception of a measurement start instruction from a user until the end of the final LFCT measurement as compared with a case where the second LFCT measurement ends after the predetermined, unchanged over-time observation time T3 elapses.

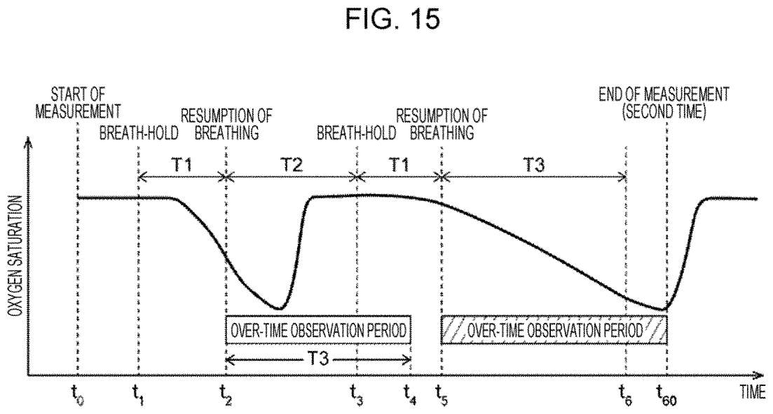

On the other hand, FIG. 15 is a graph illustrating a temporal change example of the oxygen saturation of the test subject in a case where the inflection point of the oxygen saturation of the test subject is detected after the unchanged over-time observation time T3 during the second LFCT measurement elapses from time t₅, at which the test subject resumes breathing.

As illustrated in FIG. 15, in a case where time t₆₀, at which the inflection point of the oxygen saturation appears, is after time t₆, at which the unchanged over-time observation time T3 elapses from time t₅, the over-time observation time T3 is extended to the period from time t₅ until time t₆₀. In the example illustrated in FIG. 15, the extended over-time observation time T3 is hatched.

When breath is repeatedly held, the inflection point of the oxygen saturation during the subsequent LFCT measurement may appear later than the inflection point of the oxygen saturation during the previous LFCT measurement.

That is, the biological information measurement apparatus 10 according to the second exemplary embodiment does not have to cancel the final LFCT measurement even if the inflection point of the oxygen saturation appears after the unchanged over-time observation time T3 elapses during the final LFCT measurement. Thus, the LFCT of the test subject may be measured accurately as compared with a case where the length of the over-time observation time T3 is not adjusted.

Note that the second exemplary embodiment has described a control example in which the period from when the test subject resumes breathing until the inflection point of the oxygen saturation is detected during the final LFCT measurement is the over-time observation time T3. The CPU 21 of the biological information measurement apparatus 10 may apply such control in which the over-time observation time T3 is changed in accordance with the detection of the inflection point of the oxygen saturation to another LFCT measurement that is different from the final LFCT measurement. In this case, the user may set the measurement in which the over-time observation time T3 is changed in accordance with the detection of the inflection point of the oxygen saturation.

### Modification Examples

Now, various modification examples of the first exemplary embodiment and the second exemplary embodiment will be described.

The CPU 21 of the biological information measurement apparatus 10 may vary the breath-adjusting time T2 in accordance with the measurement status of the LFCT of the test subject instead of using a fixed value as the breath-adjusting time T2.

For example, in a case where the test subject repeatedly holds their breath, even if breath-hold times are equal, the test subject tends to feel more difficult to hold their breath over time. Thus, the CPU 21 may perform control such that the breath-adjusting time T2 is adjusted to be longer as the LFCT measurement on the same test subject approaches the final measurement.

As the test subject feels more difficult to breathe, the oxygen saturation at the end of the fixed breath-adjusting time T2, that is, the oxygen saturation before a breath-hold, may fall. Thus, for each LFCT measurement, the CPU 21 may set an extension rate of the breath-adjusting time T2 by using a decrease rate of the oxygen saturation before a breath-hold during a previous LFCT measurement. For example, the CPU 21 may set the decrease rate of the oxygen saturation before a breath-hold during a previous LFCT measurement as the extension rate of the breath-adjusting time T2 during a subsequent LFCT measurement.

Note that the CPU 21 does not necessarily set the breath-adjusting time T2 during each LFCT measurement to be longer than the breath-adjusting time T2 in a previous LFCT measurement. For example, before a predetermined non-final LFCT measurement (referred to as “non-final measurement”), the breath-adjusting time T2 during each measurement may be equal, and after the non-final measurement, the breath-adjusting time T2 during each measurement may be made longer than the breath-adjusting time T2 in each LFCT measurement at or before the non-final measurement.

In addition, instead of using a fixed value as the breath-hold time T1, the CPU 21 may vary the breath-hold time T1 in accordance with the LFCT measurement status of the test subject.

As described above, the test subject tends to feel more difficult to hold their breath as the measurement approaches the final LFCT measurement. Although the length of the breath-adjusting time T2 is adjusted in the above example for this, the CPU 21 may perform control such that the breath-hold time T1 is adjusted to be shorter as the LFCT measurement on the same test subject approaches the final measurement.

In this case, for each LFCT measurement, the CPU 21 may set the shortening rate of the breath-hold time T1 by using a normal decrease rate of the oxygen saturation during a previous LFCT measurement. For example, the CPU 21 may set the decrease rate of the oxygen saturation before a breath-hold during a previous LFCT measurement as the shortening rate of the breath-hold time T1 during a subsequent LFCT measurement.

As described above, the time from when the test subject resumes breathing after a breath-hold until the inflection point of the oxygen saturation appears differs depending on test subject. The value of the oxygen saturation at the inflection point also differs depending on test subject, and there are differences. Therefore, the breath-hold time T1 is set to a length such that the value of the oxygen saturation at the inflection point of as many test subjects as possible falls below the reference value H of the oxygen saturation.

However, among test subjects, there is a test subject for whom the value of the oxygen saturation at the inflection point does not fall below the reference value H of the oxygen saturation, for example, due to changes in physical condition. For such a test subject, the accuracy of the measured LFCT may be lower than the accuracy of the LFCT measured in a state where the value of the oxygen saturation at the inflection point falls below the reference value H of the oxygen saturation.

On the other hand, among test subjects, there is a test subject for whom the value of the oxygen saturation at the inflection point falls below the reference value H of the oxygen saturation more than necessary. For such a test subject, the accuracy of the measured LFCT may be the same as the accuracy of the LFCT measured in a state where the value of the oxygen saturation at the inflection point is the same as the reference value H of the oxygen saturation.

Such a value of the oxygen saturation at the inflection point varies depending on the length of the breath-hold time. Thus, the CPU 21 may perform control such that the breath-hold time T1 is varied depending on the difference between the lowest value of the oxygen saturation of the test subject, that is, the value of the oxygen saturation at the inflection point, and the reference value H of the oxygen saturation.

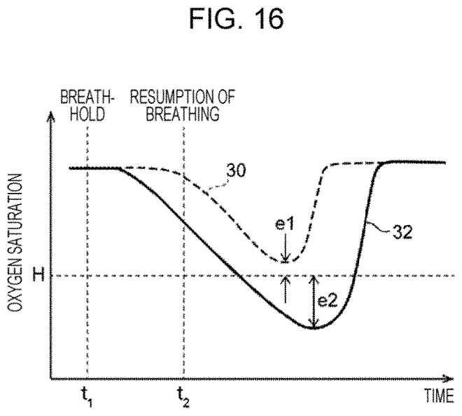

FIG. 16 illustrates an example of the difference between the value of the oxygen saturation at the inflection point and the reference value H of the oxygen saturation. In FIG. 16, an oxygen saturation curve 30 illustrates an example in which the value of the oxygen saturation at the inflection point does not fall below the reference value H of the oxygen saturation, and an oxygen saturation curve 32 illustrates an example in which the value of the oxygen saturation at the inflection point falls below the reference value H of the oxygen saturation. In addition, a difference e1 represents the difference between the inflection point on the oxygen saturation curve 30 and the reference value H of the oxygen saturation, and a difference e2 represents the difference between the inflection point on the oxygen saturation curve 32 and the reference value H of the oxygen saturation.

The CPU 21 sets the breath-hold time T1 to be longer than the preset breath-hold time T1 for a test subject for whom, as in the oxygen saturation curve 30, the value of the oxygen saturation at the inflection point does not fall below the reference value H of the oxygen saturation. As a result, as the difference between the value of the oxygen saturation at the inflection point and the reference value H of the oxygen saturation is larger, the inflection point of the oxygen saturation may fall below the reference value H of the oxygen saturation. Since the breath-hold time T1 is made longer than the preset breath-hold time T1, the LFCT measurement accuracy is improved as compared with a case where the breath-hold time T1 is not extended.

The CPU 21 also sets the breath-hold time T1 to be shorter than the preset breath-hold time T1 for a test subject for whom, as in the oxygen saturation curve 32, the value of the oxygen saturation at the inflection point does not fall below the reference value H of the oxygen saturation more than necessary. As a result, as the difference between the value of the oxygen saturation at the inflection point and the reference value H of the oxygen saturation is larger, the inflection point of the oxygen saturation may fall below the reference value H of the oxygen saturation and the inflection point of the oxygen saturation may approach the reference value H of the oxygen saturation. Since the breath-hold time T1 is made shorter than the preset breath-hold time T1, while the LFCT measurement accuracy is maintained, the time for measuring the LFCT is shortened as compared with a case where the breath-hold time T1 is not shortened.

Aspects of the biological information measurement apparatus 10 have been described above in the exemplary embodiments. However, the disclosed aspects of the biological information measurement apparatus 10 are examples, and aspects of the biological information measurement apparatus 10 are not limited to the scope described in the exemplary embodiments. Various modifications or improvements may be made for the exemplary embodiments without departing from the gist of the present disclosure, and aspects with the modifications or improvements are also included in the technical scope of the present disclosure. For example, the orders in the biological information measurement process described in the first exemplary embodiment and the second exemplary embodiment may be changed without departing from the gist of the present disclosure.

In addition, the above exemplary embodiments have described a case where software implements the biological information measurement process as an example. However, hardware may implement a process equivalent to the biological information measurement process illustrated in FIG. 9 and FIG. 10 and the biological information measurement process illustrated in FIG. 9 and FIG. 13. In such a case, the process speed may be increased compared with a case where software implements the biological information measurement process.

The above exemplary embodiments have described an example in which the biological information measurement program is stored in the ROM 22. However, the biological information measurement program is not necessarily stored in the ROM 22. The biological information measurement program according to an exemplary embodiment of the present disclosure may be provided by being recorded on a storage medium readable by the computer 20. For example, the biological information measurement program may be provided by being recorded on an optical disc such as a compact disc read only memory (CD-ROM) or a digital versatile disc read only memory (DVD-ROM). In addition, the biological information measurement program may also be provided by being recorded on a portable semiconductor memory such as a universal serial bus (USB) memory or a memory card.

The ROM 22, the non-volatile memory 24, the CD-ROM, the DVD-ROM, the USB memory, and the memory card are examples of a non-transitory computer readable medium.

Furthermore, the biological information measurement apparatus 10 may download the biological information measurement program from an external apparatus that is connected to the communication unit 29 via a communication line and may store the downloaded biological information measurement program in a non-transitory computer readable medium. In this case, the CPU 21 of the biological information measurement apparatus 10 reads, from the non-transitory computer readable medium, the biological information measurement program, which is downloaded from the external apparatus, and executes the biological information measurement process.

### Third Exemplary Embodiment

Now, referring back to FIG. 1, a method of measuring blood flow information and oxygen saturation in blood, which are examples of biological information, particularly biological information related to blood, will be described.

FIG. 1 is a schematic diagram illustrating an example of measuring blood flow information and oxygen saturation in blood according to the present exemplary embodiment. As illustrated in FIG. 1, a light emitting element 1 radiates light onto a test subject's body (living body 8). The light is reflected on or passes through the arteries 4, the veins 5, the capillaries 6, and the like, which are running through the inside of the living body 8. The light is received by the light receiving element 3, and the intensity of the light, that is, the amount of light received from reflected light or transmitted light, is used for measuring the blood flow information and the oxygen saturation in blood.

### Measurement of Blood Flow Information

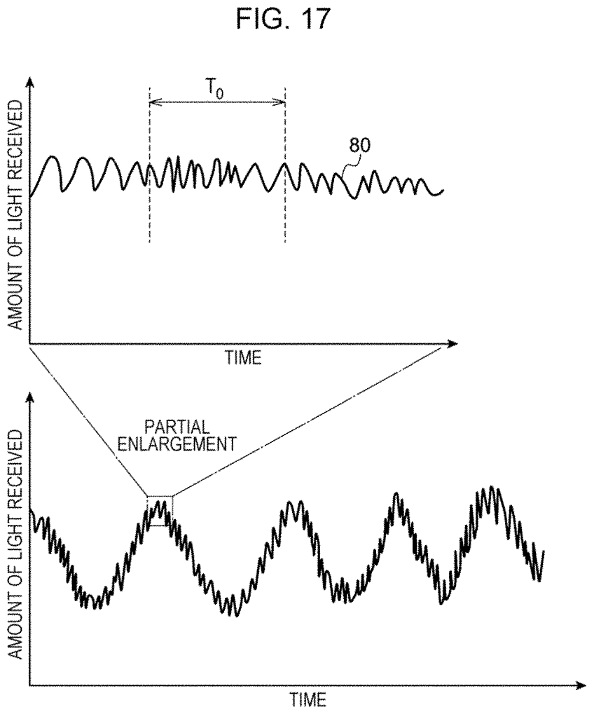

FIG. 17 is a graph illustrating an example of changes in the amount of light received from light reflected from the living body 8 according to the present exemplary embodiment.

Note that in FIG. 17, the horizontal axis of a graph 80 represents the passage of time, while the vertical axis represents the amount of light received by the light receiving element 3.

As illustrated in FIG. 17, the amount of light received by the light receiving element 3 changes over time. This is thought to be because of the influence of three optical phenomena that occur when the living body 8 including blood vessels is irradiated with light.

A first optical phenomenon is thought to be a change in the absorbance of light caused because the amount of blood existing inside the blood vessels being measured changes due to pulsation. Blood includes blood cells such as red blood cells and moves through blood vessels such as the capillaries 6. Thus, changes in the amount of blood also cause the number of blood cells moving through the blood vessels to change, which may influence the amount of light received by the light receiving element 3.

A second optical phenomenon is thought to be the influence of a Doppler shift.

FIG. 18 is a schematic diagram for describing a Doppler shift produced in a case where blood vessels are irradiated with laser light according to the present exemplary embodiment.

As illustrated in FIG. 18, in a case where, for example, the light emitting element 1 radiates coherent light 40 of a frequency ω₀, such as laser light, onto a region including the capillaries 6, which is an example of blood vessels, scattered light 42 scattered by blood cells moving through the capillaries 6 produces a Doppler shift having a difference frequency Δω₀ determined by the movement speed of the blood cells. On the other hand, the frequency of the scattered light 42 scattered by tissue (stationary tissue) such as skin that does not include moving objects such as blood cells maintains the same frequency ω₀ as the frequency of the radiated laser light. Consequently, the frequency ω₀+Δω₀ of the laser light scattered by blood vessels such as the capillaries 6 and the frequency ω₀ of the laser light scattered by stationary tissue interfere with each other, a beat signal having the difference frequency Δω₀ is observed by the light receiving element 3, and the amount of light received by the light receiving element 3 changes over time. Note that the difference frequency Δω₀ of the beat signal observed by the light receiving element 3 depends on the movement speed of the blood cells, but is included in a range with an upper limit of approximately a few dozen kHz.

A third optical phenomenon is thought to be the influence of a speckle.

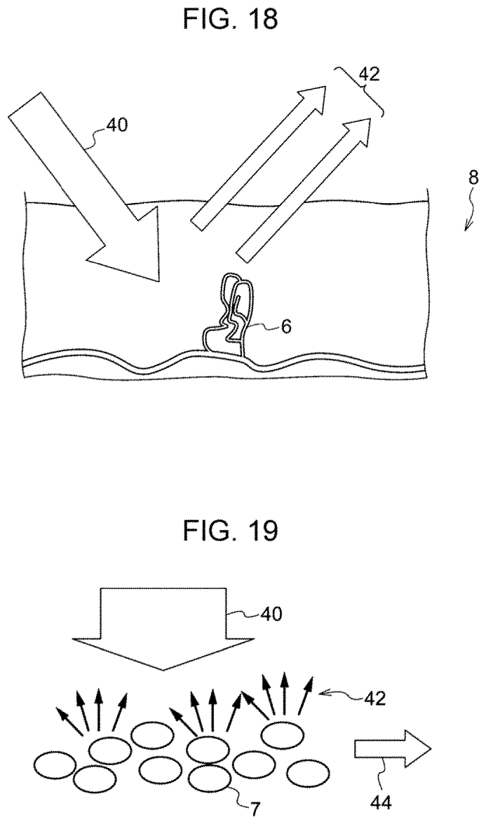

FIG. 19 is a schematic diagram for describing a speckle produced in a case where a blood vessel is irradiated with laser light according to the present exemplary embodiment.

As illustrated in FIG. 19, in a case where the light emitting element 1 radiates the coherent light 40, such as laser light, onto blood cells 7 such as red blood cells moving through a blood vessel in the direction of an arrow 44, laser light colliding with the blood cells 7 scatters in various directions. Since the scattered light has different phases, the scattered light interferes with itself randomly. Consequently, a random light intensity distribution having a spotted pattern is produced. A distribution pattern of light intensity formed in this way is called “speckle pattern”.

As described above, since the blood cells 7 move through a blood vessel, the state of light scattering in the blood cells 7 changes, and the speckle pattern varies over time. Consequently, the amount of light received by the light receiving element 3 changes over time.

Next, an example of how to obtain blood flow information will be described. In a case where the amount of light received by the light receiving element 3 over time illustrated in FIG. 17 is obtained, data included in the range of a predetermined unit time T₀ is cut out and subjected to the fast Fourier transform (FFT), for example. Thus, a spectral distribution by frequency ω is obtained.

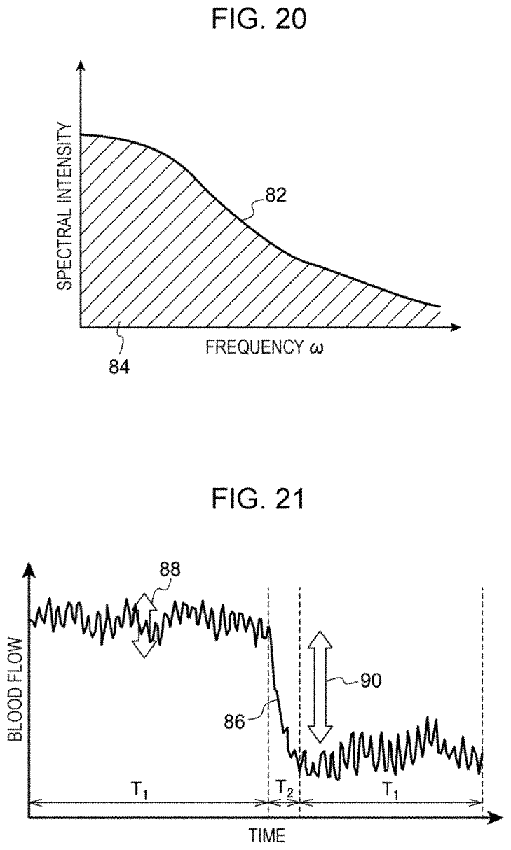

FIG. 20 is a graph illustrating an example of the spectral distribution by frequency ω in the unit time T₀ according to the present exemplary embodiment. Note that in FIG. 20, the horizontal axis of a graph 82 represents the frequency ω, while the vertical axis represents the spectral intensity.

Here, the amount of blood is proportional to a value obtained by standardizing the area of the power spectrum illustrated in a shaded region 84 enclosed by the horizontal axis and the vertical axis of the graph 82 by the total amount of light. Also, the blood flow speed is proportional to the average value of the frequency of the power spectrum expressed by the graph 82, and thus is proportional to the value obtained by taking the value of integrating the product of the frequency ω and the power spectrum for the frequency ω over the frequency ω, and dividing the integral value by the area of the shaded region 84.

Note that the amount of blood flow is expressed as the product of the amount of blood and the blood flow speed and thus may be calculated according to a formula of the amount of blood and the blood flow speed above. The amount of blood flow, the blood flow speed, and the amount of blood are examples of blood flow information, but the blood flow information is not limited thereto.

FIG. 21 is a graph illustrating an example of changes in the amount of blood flow per unit time T₀ according to the present exemplary embodiment. Note that in FIG. 21, the horizontal axis of a graph 86 represents time, while the vertical axis represents the amount of blood flow.

As illustrated in FIG. 21, the amount of blood flow varies with time, and the trend of variations is classified into two types. For example, as compared with a variation range 88 of the amount of blood flow in a segment T₁ of FIG. 21, a variation range 90 of the amount of blood flow in a segment T₂ is large. The reason for this is thought to be that, while the change in the amount of blood flow in the segment T₁ is the change in the amount of blood flow mostly associated with pulsation, the change in the amount of blood flow in the segment T₂ indicates a change in the amount of blood flow associated with a cause such as congestion, neural activity, or the like, for example.

### Measurement of Oxygen Saturation

Next, the measurement of the oxygen saturation in blood will be described. The oxygen saturation in the blood is an example of the blood oxygen concentration, and is an indicator indicating how much hemoglobin in the blood is bonded to oxygen. As the oxygen saturation in the blood falls, symptoms such as anemia occur more readily.

FIG. 2 is to be referred to again.

FIG. 2 is a graph illustrating an example of changes in the amount of light absorbed by the living body 8 according to the present exemplary embodiment. Note that in FIG. 2, the horizontal axis of the graph represents time, while the vertical axis represents the amount of light absorbed.

As illustrated in FIG. 2, the amount of light absorbed in the living body 8 tends to vary over time.

Further examination of the variations in the amount of light absorbed by the living body 8 reveals that the amount of light absorbed by the arteries 4 varies widely, whereas for the veins 5 and other tissue including stationary tissue, the amount of variation is small enough to consider that there are no variations in the amount of light absorbed as compared with the amount of light absorbed by the arteries 4. This is because arterial blood that the heart pumps out moves through blood vessels in association with pulse waves to cause the arteries 4 to expand and contract over time in the cross-sectional direction of the arteries 4, and the thickness of the arteries 4 change. Note that the range indicated by the arrow 94 in FIG. 2 denotes the amount of variation in the amount of light absorbed corresponding to the change in the thickness of the arteries 4.

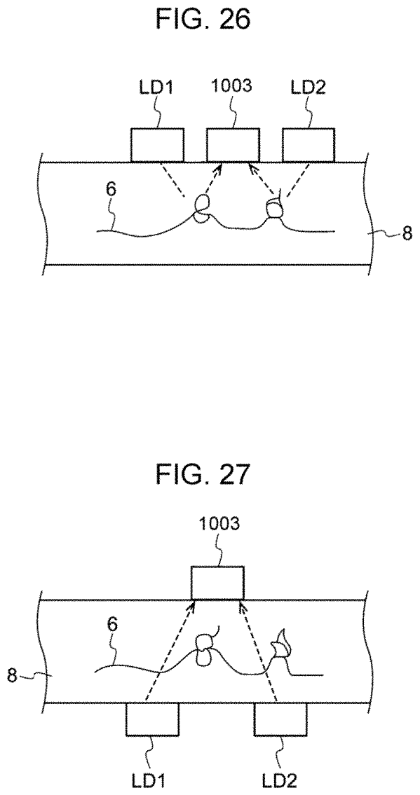

Now, FIG. 26 and FIG. 27 are to be temporarily referred to. FIG. 26 illustrates an example of arrangement of light emitting elements and a light receiving element in the biological information measurement apparatus according to the third exemplary embodiment, and FIG. 27 illustrates another example of arrangement of the light emitting elements and the light receiving element in the biological information measurement apparatus according to the third exemplary embodiment.

Note that when it is necessary to distinguish a light emitting element 1 that radiates IR light from a light emitting element 1 that radiates red light, the light emitting element 1 that radiates IR light will be referred to as “light emitting element LD1”, while the light emitting element 1 that radiates red light will be referred to as “light emitting element LD2” in the following description. Also, as an example, the light emitting element LD1 is a light emitting element 1 used for calculation of the amount of blood flow, while the light emitting element LD1 and the light emitting element LD2 are light emitting elements 1 used for calculation of the oxygen saturation in the blood.

In addition, to measure the oxygen saturation in the blood, it is known that frequencies of about 30 Hz to about 1000 Hz are enough as a frequency of measuring the amount of light received, and thus, frequencies of about 30 Hz to about 1000 Hz are also enough as a light emission frequency that expresses the number of flashes per second by the light emitting element LD2. Consequently, in view of power consumption and the like in the light emitting element LD2, it is desirable to set the light emission frequency of the light emitting element LD2 lower than the light emission frequency of light emitting element LD1, but the light emission frequency of the light emitting element LD2 may be matched to the light emission frequency of the light emitting element LD1 to make the light emitting element LD1 and the light emitting element LD2 emit light in alternation.

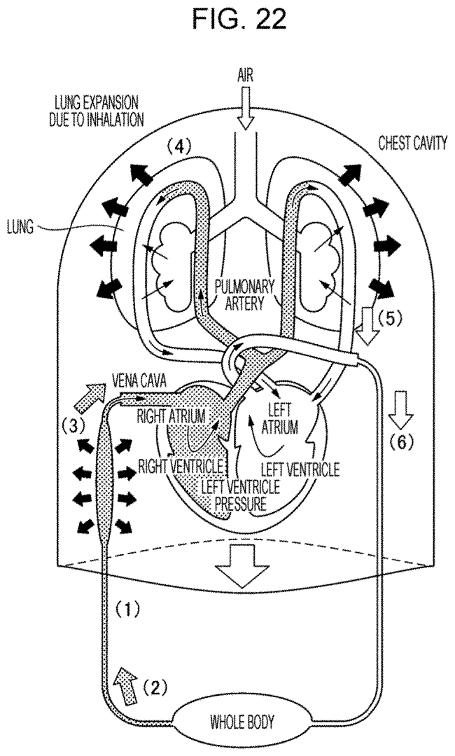

Next, referring to FIG. 22, a principle of measuring a respiration waveform from a pulse wave signal obtained from a peripheral body part of the living body 8 will be described. Examples of the peripheral body part herein include the fingertips of the hands, the tips of the toes, the earlobes, and the like. Note that the peripheral body part also includes body parts past the elbows, body parts past the knees, and the like. In addition, the respiration waveform is the waveform of a signal indicating the respiratory state of the living body 8, and is the waveform of a time-series signal expressing a temporal change in exhalation and inhalation.

FIG. 22 is a schematic diagram for describing the principle of measuring the respiration waveform according to the present exemplary embodiment. As illustrated in FIG. 22, during inhalation, the amplitude of the pulse wave signal decreases according to the steps below.

(S1) The intrathoracic pressure falls to a negative pressure, and the lungs expand.
(S2) The amount of venous return increases.
(S3) The amount of blood flowing into the right atrium increases.
(S4) The vascular bed of the lungs expands, and the amount of blood retained by the lungs increases.
(S5) The amount of blood returning to the left atrium from the lungs decreases.
(S6) The stroke volume of the left ventricle decreases.
(S7) The amplitude of the pulse wave decreases.

On the other hand, during exhalation, the amplitude of the pulse wave signal increases according to the steps below.

(S8) Blood squeezed out from the lungs flows into the left ventricle.
(S9) The amplitude of the pulse wave increases.

In other words, since the influence of the “pump action of the lungs” caused by respiration is superimposed onto the pulsation caused by the “pump action of the heart”, it is possible to measure the respiration waveform from a pulse wave signal obtained from a peripheral body part of the living body 8.

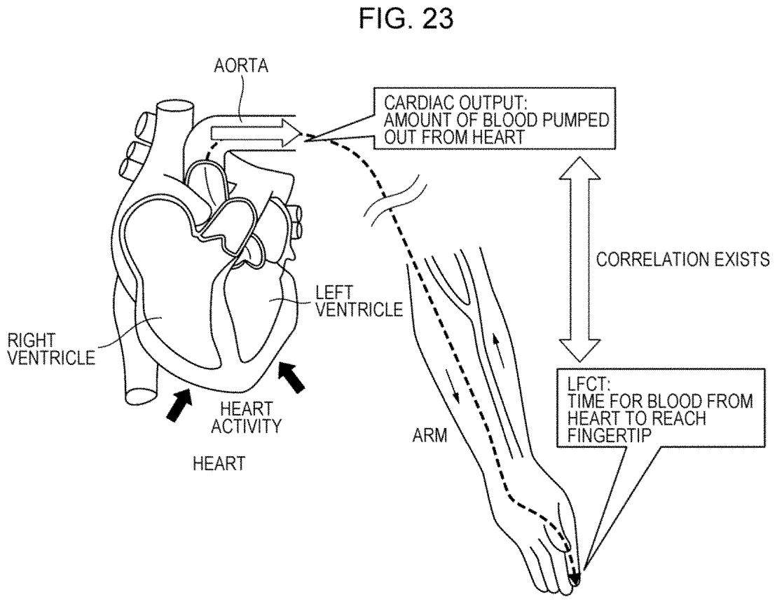

Next, referring to FIG. 23, a principle of measuring the lung to finger circulation time (LFCT), which is an example of an indicator correlated with the output of blood from the heart will be described. The output herein is not limited to the cardiac output described above, and also includes the stroke volume, cardiac index, and the like. Note that the cardiac output is defined as the amount of blood pumped to the arteries by contraction of the heart per unit time (for example, per minute). The stroke volume is defined as the amount of blood pumped to the arteries by a single contraction of the heart. The cardiac index is defined as a coefficient obtained by dividing the cardiac output by the body surface area of the test subject. Also, the LFCT is defined as the time for oxygen taken in by respiration to reach a fingertip through the lungs and heart.

FIG. 23 is a schematic diagram for describing the principle of measuring the output according to the present exemplary embodiment. As illustrated in FIG. 23, the above output and the LFCT are correlated with each other. For example, if CO is the cardiac output, which is an example of the output, the cardiac output CO is calculated according to Formula (6) below.

CO=(a₀×S)/LFCT (6)

Herein, a₀ is a constant. For example, a₀=50 is used. Also, S is the body surface area (m²) of the test subject, and the units of the LFCT are seconds.

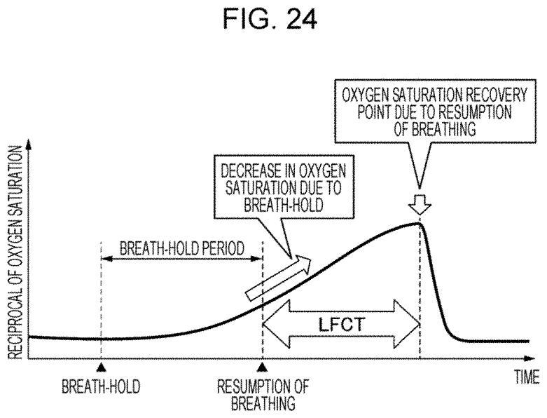

FIG. 24 is a graph for describing an example of a method of measuring the LFCT according to the present exemplary embodiment. Note that in FIG. 24, the vertical axis represents the reciprocal of the oxygen saturation, while the horizontal axis represents time.

As illustrated in FIG. 24, the LFCT according to the present exemplary embodiment is measured from the change in the oxygen saturation described above. In other words, the LFCT is obtained by measuring the time from the point in time at which a test subject who resumes breathing after a breath-hold for a fixed period, until the inflection point that indicates that the oxygen saturation has recovered.

As described above, the simplest way to change the test subject's blood oxygen concentration is that the test subject holds their breath. However, for the uncomfortableness of holding their breath, they may hold their breath with much air inhaled in their lungs. In this case, a waveform pattern representing the change in the blood oxygen concentration becomes inappropriate, from which an inflection point in the waveform pattern generated by the change in the blood oxygen concentration is not to be specified. The existing technology has required a complex algorithm in order to determine whether the waveform pattern is appropriate, imposing a heavy burden on the determination processing. Thus, a simple determination method has been desired.

The present exemplary embodiment will describe a biological information measurement apparatus that determines that a waveform pattern is appropriate in a more simplified manner than in a case of determining whether a waveform pattern is appropriate by using an existing algorithm.

The present exemplary embodiment focuses on the fact that there is only a change in the output due to pulsation (amount of blood) and a correlation between pulse waves due to two wavelengths (e.g., IR light signal and red light signal) to be measured is high if there is no change in the blood oxygen concentration and that the correlation is low if there is a change in the blood oxygen concentration. In other words, if the correlation between two pulse waves is high, the waveform pattern representing the change in the blood oxygen concentration is determined to be inappropriate, and if the correlation is low, the waveform pattern representing the change in the blood oxygen concentration is determined to be appropriate.

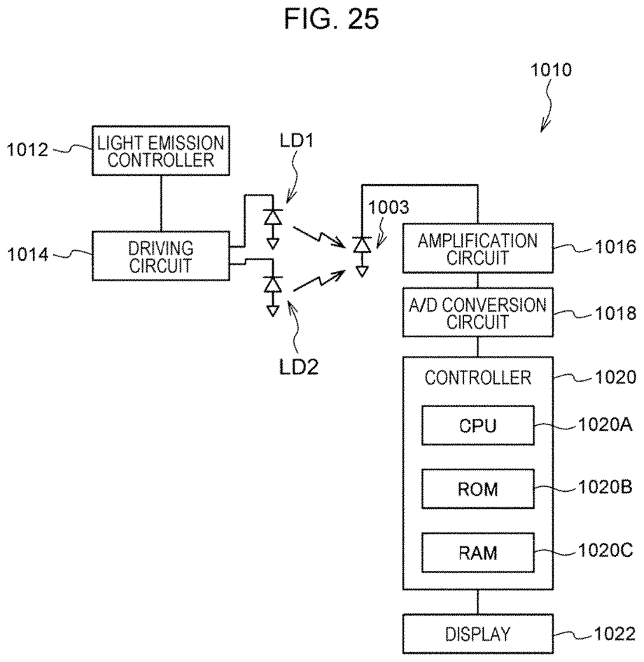

FIG. 25 is a block diagram illustrating an example of an electrical configuration of a biological information measurement apparatus 1010 according to the present exemplary embodiment.

As illustrated in FIG. 25, the biological information measurement apparatus 1010 according to the present exemplary embodiment includes a light emission controller 1012, a driving circuit 1014, an amplification circuit 1016, an analog/digital (A/D) conversion circuit 1018, a controller 1020, a display 1022, the light emitting element LD1, the light emitting element LD2, and a light receiving element 1003. Note that the light emitting element LD1, the light emitting element LD2, the light receiving element 1003, and the amplification circuit 1016 form a sensor unit. Also, the light emission controller 1012, the driving circuit 1014, the amplification circuit 1016, the A/D conversion circuit 1018, the controller 1020, and the display 1022 form a principal unit. In the present exemplary embodiment, the sensor unit and the principal unit are formed as separate units that are communicable in a wired or wireless manner. However, the sensor unit and the principal unit may also be formed as a single unit. Also, the sensor unit is attached to adhere closely to the living body 8 such that external light is not input. As an example, the sensor unit according to the present exemplary embodiment is attached to a fingertip of the living body 8, but is also attachable to another peripheral body part such as an earlobe.

The light emission controller 1012 outputs a control signal that controls a light emission cycle and a light emission period of the light emitting element LD1 and the light emitting element LD2 to the driving circuit 1014 including a power supply circuit that supplies driving power to the light emitting element LD1 and the light emitting element LD2. Note that the light emission controller 1012 may also be implemented as part of the controller 1020.

Upon receipt of the control signal from the light emission controller 1012, in accordance with the light emission cycle and light emission period indicated by the control signal, the driving circuit 1014 supplies driving power to the light emitting element LD1 and the light emitting element LD2 and drives the light emitting element LD1 and the light emitting element LD2.

The light receiving element 1003 receives light of a first wavelength from the light emitting element LD1 and outputs a first received light signal corresponding to the received light of the first wavelength, and also receives light of a second wavelength from the light emitting element LD2 and outputs a second received light signal corresponding to the received light of the second wavelength. Note that in the present exemplary embodiment, a range of wavelengths corresponding to the infrared region is applied as the first wavelength, and a range of wavelengths corresponding to the red region is applied as the second wavelength. Also, an IR light signal is applied as the first received light signal, and a red light signal is applied as the second received light signal.

The amplification circuit 1016 converts a current corresponding to the light intensity produced by the light receiving element 1003 into a voltage, and amplifies the voltage to a voltage level prescribed as an input voltage range of the A/D conversion circuit 1018.

The A/D conversion circuit 1018 receives the voltage amplified by the amplification circuit 1016 as input, and outputs an amount of light received by the light receiving element 1003 expressed by the magnitude of the voltage as a numerical value.

The controller 1020 includes a central processing unit (CPU) 1020A, a read only memory (ROM) 1020B, and a random access memory (RAM) 1020C. The ROM 1020B stores the biological information measurement program. The biological information measurement program may be preinstalled in the biological information measurement apparatus 1010, for example. The biological information measurement program may also be implemented by being stored in a non-volatile storage medium or distributed over a network, and installed in the biological information measurement apparatus 1010 as appropriate. Note that anticipated examples of the non-volatile storage medium include a compact disc read only memory (CD-ROM), a magneto-optical disc, an HDD, a digital versatile disc read only memory (DVD-ROM), a flash memory, a memory card, and the like.

The display 1022 displays a result of measuring biological information. For the display 1022, for example, a liquid crystal display (LCD), an organic electroluminescent (EL) display, or the like is used. The display 1022 includes an integrated touch panel.

FIG. 26 illustrates an arrangement example of the light emitting element LD1, the light emitting element LD2, and the light receiving element 1003 in the biological information measurement apparatus 1010 according to the present exemplary embodiment. FIG. 27 illustrates another arrangement example of the light emitting element LD1, the light emitting element LD2, and the light receiving element 1003 in the biological information measurement apparatus 1010 according to the present exemplary embodiment.

As illustrated in FIG. 26, the light emitting element LD1, the light emitting element LD2, and the light receiving element 1003 are arranged side by side in one direction to face the surface of the living body 8. In this case, the light receiving element 1003 receives light from the light emitting element LD1 and the light emitting element LD2 that has been transmitted through the surface of the living body 8 and the vicinity thereof.

Note that the arrangement of the light emitting element LD1, the light emitting element LD2, and the light receiving element 1003 is not limited to the arrangement example in FIG. 26. For example, as illustrated in FIG. 27, the light emitting element LD1, the light emitting element LD2, and the light receiving element 1003 may also be arranged such that the light emitting elements LD1 and LD2 face the light receiving element 1003 with the living body 8 sandwiched between the light emitting elements LD1 and LD2 and the light receiving element 1003. In this case, the light receiving element 1003 receives light from the light emitting element LD1 and the light emitting element LD2 that has been transmitted through the living body 8.

Here, as an example, each of the light emitting element LD1 and the light emitting element LD2 will be described as a surface-emitting laser element. However, each of the light emitting element LD1 and the light emitting element LD2 is not limited to a surface-emitting laser element and may be an edge-emitting laser element. Also, the light radiated from each of the light emitting element LD1 and the light emitting element LD2 does not have to be laser light. In this case, a light-emitting diode (LED) or an organic light-emitting diode (OLED) may be used for each of the light emitting element LD1 and the light emitting element LD2.

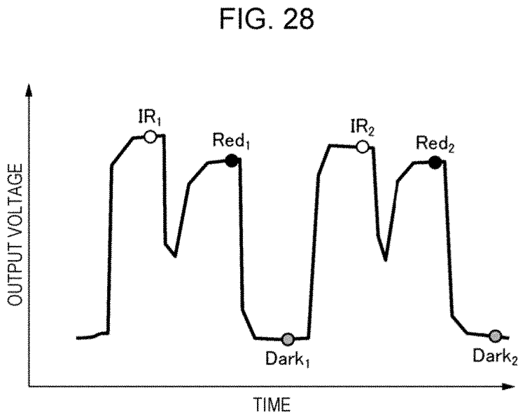

FIG. 28 is a graph illustrating an example of sampling timings of data in the light receiving element 1003 according to the present exemplary embodiment. In FIG. 28, the positions of the circle marks indicate sampling timings. Note that in FIG. 28, the vertical axis represents an output voltage of the light receiving element 1003, while the horizontal axis represents time.

As illustrated in FIG. 28, if output voltages corresponding to light that the light receiving element 1003 receives from the light emitting element LD1 are then IR(t)=IR₁, IR₂, . . . , IRₙ is obtained as time series data. Similarly, if output voltages corresponding to light that the light receiving element 1003 receives from the light emitting element LD2 are Red₁, Red₂, . . . , Redₙ, then Red(t)=Red₁, Red₂, . . . , Redₙ, is obtained as time series data. At this time, periods during which neither of the light emitting element LD1 and the light emitting element LD2 emit light may also be provided, and outputs Dark₁, Dark₂, . . . , Darkₙ, may be obtained in these dark states. In this case, IR(t) may also be IR₁-Dark₁, IR₂-Dark₂, . . . , IRₙ-Darkₙ. Similarly, Red(t) may also be Red₁-Dark₁, Red₂-Dark₂, . . . Redₙ-Darkₙ. It is desirable for the above data to be sampled near the end of each light emission period in a state of stable output.

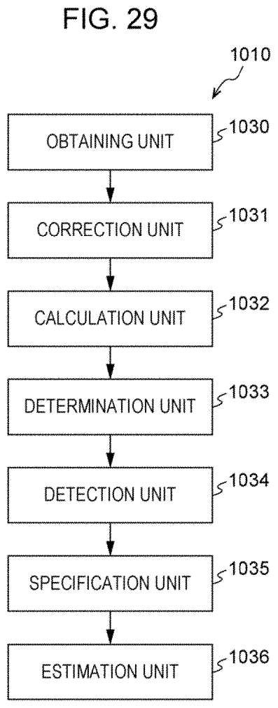

The CPU 1020A of the biological information measurement apparatus 1010 according to the present exemplary embodiment loads the biological information measurement program stored in the ROM 1020B into the RAM 1020C and executes the program, and thereby functions as each unit illustrated in FIG. 29. Note that the CPU 1020A is an example of a processor.

FIG. 29 is a block diagram illustrating an example of a functional configuration of the biological information measurement apparatus 1010 according to the third exemplary embodiment.

As illustrated in FIG. 29, the CPU 1020A of the biological information measurement apparatus 1010 according to the present exemplary embodiment functions as an obtaining unit 1030, a correction unit 1031, a calculation unit 1032, a determination unit 1033, a detection unit 1034, a specification unit 1035, and an estimation unit 1036.

The obtaining unit 1030 obtains each of the IR light signal and the red light signal output from the light receiving element 1003. In this case, the IR light signal is an example of a first signal, and the red light signal is an example of a second signal.

The correction unit 1031 corrects the IR light signal, for example, by multiplying the value of the IR light signal by a coefficient to reduce the difference between the amount of change in the IR light signal (hereinafter designated as ΔIR) and the amount of change in the red light signal (hereinafter designated as ΔRed) associated with a change in the amount of arterial blood in the living body 8. The change in the amount of arterial blood expresses the amplitude of pulsation associated with heartbeat. Note that the target to be corrected may also be the value of the red light signal.

It is desirable for the above correction to make ΔIR and ΔRed equal. Herein, ΔIR is expressed as the amplitude of the IR light signal, and ΔRed is expressed as the amplitude of the red light signal. In this case, the above correction is performed by multiplying the values of the IR light signal (IR(t)) by a coefficient α representing the amplitude ratio of ΔIR and ΔRed (ΔRed/ΔIR). In other words, the corrected output of IR(t) is α×IR(t).

The calculation unit 1032 calculates a waveform pattern representing the change in the blood oxygen concentration in the living body 8 on the basis of the IR light signal and the red light signal, either of which is corrected by the correction unit 1031. As an example, the waveform pattern representing the change in the blood oxygen concentration is expressed as the difference between the IR light signal and the red light signal, either of which is corrected by the correction unit 1031 (hereinafter, this difference is designated as “pulse wave difference”). For example, if the pulse wave difference is β(t), β(t) is obtained according to Formula (7) below.

β(t)=α×IR(t)−Red(t) (7)

The determination unit 1033 determines that the pulse wave difference 13(t) calculated by the calculation unit 1032 is appropriate if the value representing a degree of correlation between the IR light signal and the red light signal is less than a threshold. Note that as the value representing the degree of correlation, for example, a coefficient of determination (=R²) is used. The coefficient of determination is an indicator indicating how well a regression line fits, the regression line being obtained from a scatter diagram in which the values (e.g., voltage values) of the IR light signal and the values (e.g., voltage values) of the red light signal for a predetermined period are plotted. The coefficient of determination is obtained by a known method. The coefficient of determination is a value of greater than or equal to 0 and less than or equal to 1 and indicates that the correlation between the IR light signal and the red light signal is higher as the value is closer to 1. In addition, the threshold is, for example, 0.8, preferably 0.7. On the other hand, if the value representing the degree of correlation is greater than or equal to the threshold, the determination unit 1033 determines that the pulse wave difference β(t) is inappropriate and gives an alarm that recommends remeasurement. For example, the alarm includes a message that recommends remeasurement in which a test subject breathes out sufficiently before a breath-hold.

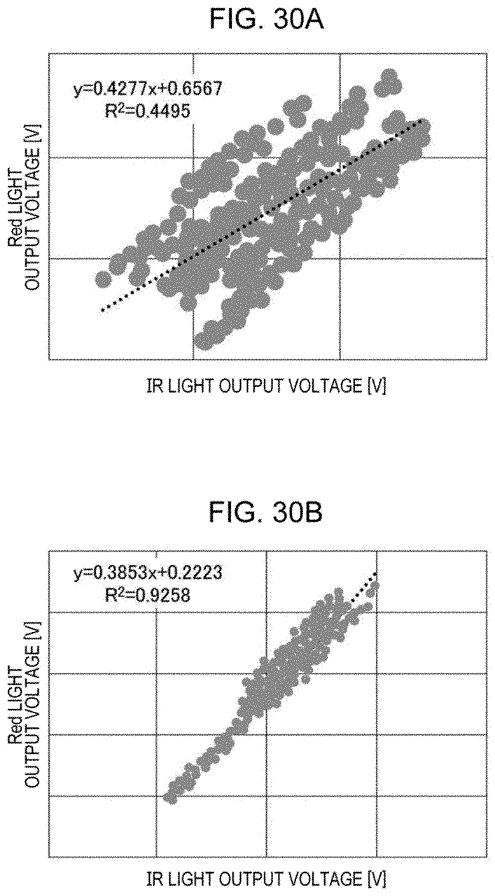

FIG. 30A is a scatter diagram illustrating an example of a correlation between the IR light output voltage and the red light output voltage in a case where there is a change in the blood oxygen concentration. FIG. 30B is a scatter diagram illustrating an example of a correlation between the IR light output voltage and the red light output voltage in a case where there is no change in the blood oxygen concentration. In FIG. 30A and FIG. 30B, the vertical axis represents the output voltage of the red light signal, while the horizontal axis represents the output voltage of the IR light signal.

In the scatter diagram in FIG. 30A, if the blood oxygen concentration changes, in other words, if the oxygen saturation falls, the correlation between the IR light signal and the red light signal is low, and the coefficient of determination (in this example, R²=0.4995) is less than the threshold (e.g., 0.8). In this case, it is determined that the pulse wave difference β(t) is appropriate. On the other hand, in the scatter diagram in FIG. 30B, if the blood oxygen concentration does not change, in other words, if the oxygen saturation does not fall, the correlation between the IR light signal and the red light signal is high, and the coefficient of determination (in this example, R²=0.9258) is greater than or equal to the threshold (e.g., 0.8). In this case, it is determined that the pulse wave difference β(t) is inappropriate.

The value representing the degree of correlation is calculated from the IR light signal and the red light signal during a predetermined time (e.g., 30 seconds) after the amount of oxygen inhaled by the living body 8 is changed (after breathing is resumed). Note that the predetermined period is desirably a period of two or more heartbeats of the living body 8 because an accurate correlation is unlikely to be obtained with a single heartbeat.

The detection unit 1034 detects the inflection point of the blood oxygen concentration associated with a change in the amount of oxygen inhaled by the living body 8 on the basis of the pulse wave difference β(t) that is determined to be appropriate by the determination unit 1033. Note that an example of a method for causing the amount of inhaled oxygen to change is the method of holding their breath and the like. Also, the change in the amount of inhaled oxygen herein is assumed to be a change that induces a change in the blood oxygen concentration for at least several seconds, and does not include slight changes due to a normal respiratory state (for example, inhaling and exhaling at an ordinary rate and an ordinary depth). In other words, in the normal respiratory state, it is determined that there is no change in the amount of inhaled oxygen, whereas in a case of causing a change from the normal respiratory state by holding their breath, taking shallow breaths, inhaling gas with a high oxygen concentration, or the like, it is determined that the amount of inhaled oxygen has changed.

The specification unit 1035 specifies the time from the point in time at which the amount of oxygen inhaled by the living body 8 changes until the inflection point in the blood oxygen concentration detected by the detection unit 1034. Note that the point in time at which the amount of inhaled oxygen changes is, for example, the point in time at which the test subject resumes breathing after a breath-hold or the like. In the present exemplary embodiment, the time specified by the specification unit 1035 is the LFCT.

The estimation unit 1036 estimates the output from the LFCT specified by the specification unit 1035. For example, Formula (6) above is used to estimate the cardiac output, which is an example of the output.

Note that each of the IR light signal and the red light signal includes a component expressing change in the amount of blood due to pulsation, neural activity, and the like, and a component expressing change in the oxygen concentration due to the change in the amount of inhaled oxygen. Additionally, according to the above pulse wave difference β(t), by multiplying IR(t) by the coefficient α(=ΔRed/ΔIR) and adopting the difference between α×IR(t) and Red(t), the components expressing the change in the amount of arterial blood are canceled out, and only the components expressing the change in the oxygen concentration are extracted.

Although the coefficient α above is (ΔRed/ΔIR) to correct the IR light signal, the coefficient α may also be (ΔIR/ΔRed) to correct the red light signal. In this case, the pulse wave difference β(t) is calculated according to Formula (8) below.

β(t)=IR(t)−α×Red(t) (8)

In addition, although a case where either the IR light signal or the red light signal, which are examples of two pulse wave signals, is corrected has been described above, both the IR light signal and the red light signal may also be corrected. Furthermore, although the red light signal is subtracted from the IR light signal above, the IR light signal may also be subtracted from the red light signal. In this case, the direction of the inflection point appearing in β(t) is different.

Note that the pulse wave signal when calculating the coefficient α and the pulse wave signal to which the calculated coefficient α is applied are shifted in time. In other words, the above correction is applied by multiplying the coefficient α representing the amplitude ratio of ΔIR and ΔRed before causing the amount of inhaled oxygen to change, by IR(t) or Red(t) after causing the amount of inhaled oxygen to change. For example, it is desirable to use a pulse wave signal when the test subject is resting before a breath-hold as the pulse wave signal to use when calculating the coefficient α.

Next, referring to FIG. 31, operations of the biological information measurement apparatus 1010 according to the third exemplary embodiment will be described. Note that FIG. 31 is a flowchart illustrating an example of a process flow of the biological information measurement program according to the third exemplary embodiment.

First, in response to the biological information measurement apparatus 1010 being powered on by an operation by the test subject or a measurement technician, the biological information measurement program is launched, and each of the following steps is executed.

In step S100 of FIG. 31, the CPU 1020A acquires the amplitude (ΔIR) of the IR light signal obtained from the light receiving element 1003, and acquires the amplitude (ΔRed) of the red light signal obtained from the light receiving element 1003. In this step S100, first, each of ΔIR and ΔRed is acquired as a pulse wave amplitude while the test subject remains in a resting state.

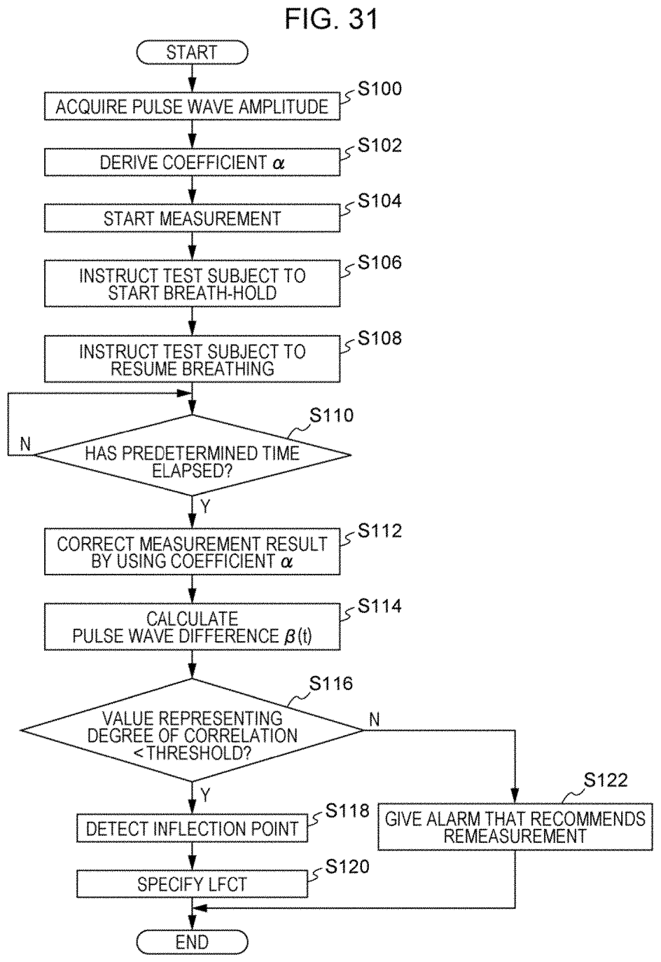

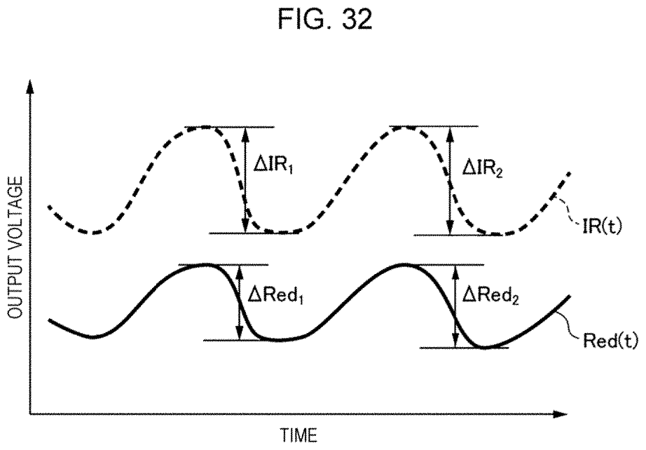

FIG. 32 is a graph illustrating an example of the amplitude of the IR light signal and the amplitude of the red light signal according to the present exemplary embodiment.

Note that in FIG. 32, the vertical axis represents the output voltage of the light receiving element 1003, while the horizontal axis represents time.

As illustrated in FIG. 32, the CPU 1020A acquires ΔIR from IR(t), which is the time series data of the values of the IR light signal, and acquires ΔRed from Red(t), which is the time series data of the values of the red light signal.

In step S102, the CPU 1020A derives the coefficient α representing the amplitude ratio of ΔIR and ΔRed on the basis of ΔIR and ΔRed acquired in step S100. As an example, the coefficient α is derived according to any of the methods below.

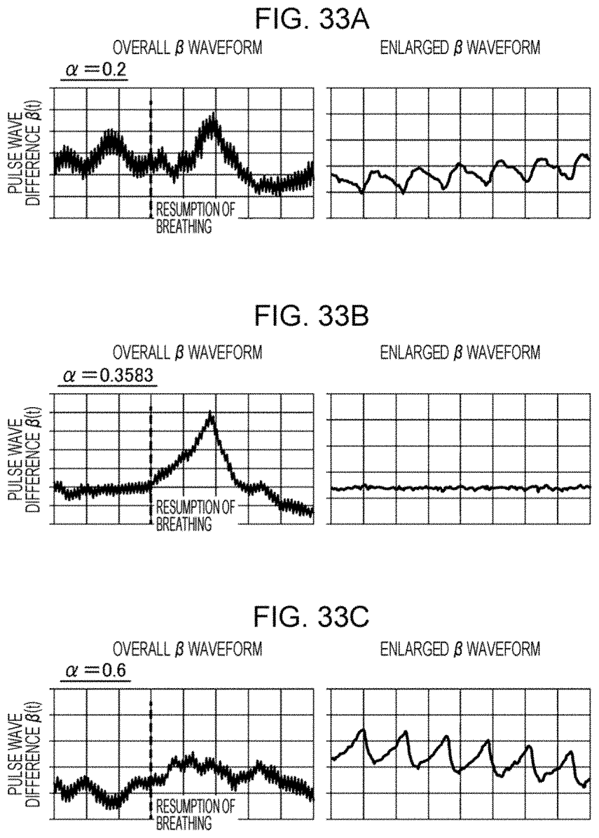

(a) The amplitude ratio obtained at any given timing is adopted. Note that in this case, the timing may also be after the start of the LFCT measurement.
(b) The average value of plural amplitude ratios obtained in a fixed period is adopted. In this method, the coefficient α that is suited to measurement is calculated as compared with a case of deriving the coefficient α by adopting the amplitude ratio at only a single point.
(c) After measurement ends, the coefficient α is changed between 0 and 1 as illustrated in FIG. 33A, FIG. 33B, and FIG. 33C, for example, and the value with the smallest frequency component of pulsation appearing in the pulse wave difference β(t) is adopted. However, in a case where the coefficient α is (ΔRed/ΔIR), the condition ΔIR>ΔRed is assumed to be satisfied. In this method, it is not necessary to derive the coefficient α during measurement, and thus, the measurement time is shortened, for example.

FIG. 33A, FIG. 33B, and FIG. 33C are graphs illustrating examples of the relationship between the coefficient α and the pulse wave difference β(t) according to the present exemplary embodiment.

Note that in FIG. 33A, FIG. 33B, and FIG. 33C, the vertical axis represents the pulse wave difference β(t). Also, in this example, α=ΔRed/ΔIR, and β(t)=α×IR(t)−Red(t).

FIG. 33A illustrates an overall waveform and an enlarged waveform of the pulse wave difference β(t) for a case where the coefficient α=0.2. The diagram on the left is the overall waveform, while the diagram on the right is the enlarged waveform.

FIG. 33B illustrates an overall waveform and an enlarged waveform of the pulse wave difference β(t) for a case where the coefficient α=0.3583. The diagram on the left is the overall waveform, while the diagram on the right is the enlarged waveform.

FIG. 33C illustrates an overall waveform and an enlarged waveform of the pulse wave difference β(t) for a case where the coefficient α=0.6. The diagram on the left is the overall waveform, while the diagram on the right is the enlarged waveform.

As the above demonstrates, in a case where the coefficient α=0.3583, the frequency component of pulsation appearing in the pulse wave difference β(t) is minimized. Consequently, according to the method of (c) above, the coefficient α=0.3583 is adopted, and the pulse wave difference β(t) in which the inflection point of oxygen concentration is at a correct position is obtained.

In step S104, the CPU 1020A receives an instruction to start measuring the LFCT while the test subject remains in a resting state. As an example, this instruction to start measurement is issued by the test subject or a measurement technician designating a measurement start through the touch panel of the display 1022 or the like.

In step S106, the CPU 1020A instructs the test subject to start a breath-hold. Specifically, for example, the CPU 1020A may cause the display 1022 to display a message such as “Please hold your breath.”, or output the instruction as speech.

In step S108, after a fixed period elapses (for example, after 20 seconds elapse) since the start of the breath-hold, the CPU 1020A instructs the test subject to resume breathing. More specifically, for example, the CPU 1020A may cause the display 1022 to display a message indicating the resumption of breathing by a countdown, or output the instruction as speech. Additionally, the fact of the resumption of breathing may also be input by an operation (such as an operation of pressing a button) by the test subject.

In step S110, the CPU 1020A determines whether a predetermined time has elapsed since the resumption of breathing. The predetermined time is preset as a duration for over-time observation and may be 60 seconds or the like, for example. Note that since the arrival time of oxygen is different depending on the measurement body part, a duration for over-time observation appropriate for the measurement body part is desirably preset. If it is determined that the predetermined time has elapsed (case of positive determination), the flow advances to step S112, whereas if it is determined that the predetermined time has not yet elapsed (case of negative determination), the flow stands by in step S110.

In step S112, the CPU 1020A performs a correction by multiplying IR(t) or Red(t) obtained through the above measurement by the coefficient α derived in step S102 above. Although the correction is performed by multiplying IR(t) by the coefficient α (ΔRed/ΔIR) in the present exemplary embodiment, setting the coefficient α to ΔIR/ΔRed suffices to correct Red(t).

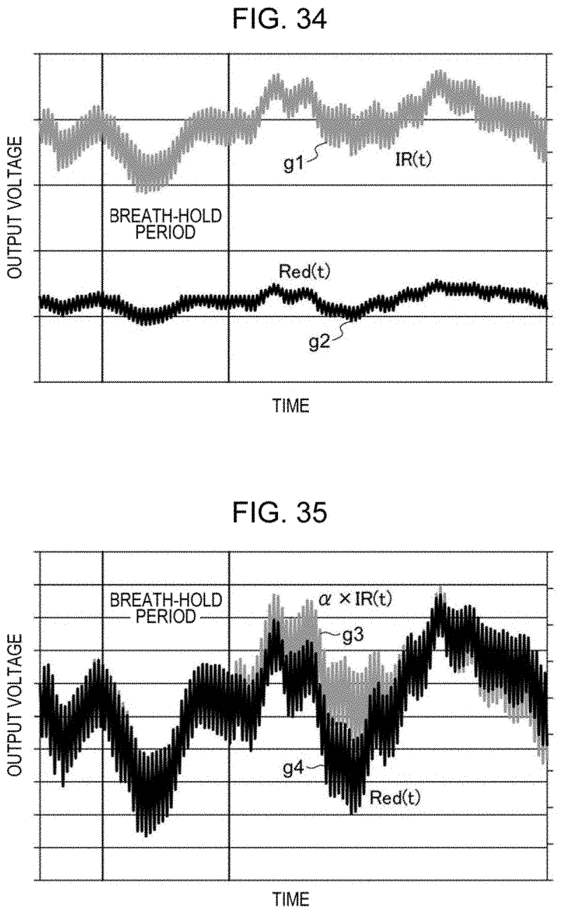

FIG. 34 is a graph illustrating an example of time series data of the IR light signal and time series data of the red light signal according to the present exemplary embodiment. Note that in FIG. 34, the vertical axis represents the output voltage of the light receiving element 1003, while the horizontal axis represents time. As illustrated in FIG. 34, a graph g1 represents IR(t), which is the time series data of the IR light signal. Also, a graph g2 represents Red(t), which is the time series data of the red light signal.

FIG. 35 is a graph illustrating an example of the time series data of the IR light signal and the time series data of the red light signal after correction according to the present exemplary embodiment. Note that in FIG. 35, the vertical axis represents the output voltage of the light receiving element 1003, while the horizontal axis represents time. As illustrated in FIG. 35, a graph g3 represents α×IR(t) obtained by multiplying IR(t) by the coefficient α and adjusting an offset. Also, a graph g4 represents Red(t), which is the time series data of the red light signal.

Note that the breathing-resumption instruction in step S108 above may also be issued if a decrease in the blood oxygen concentration is detected.

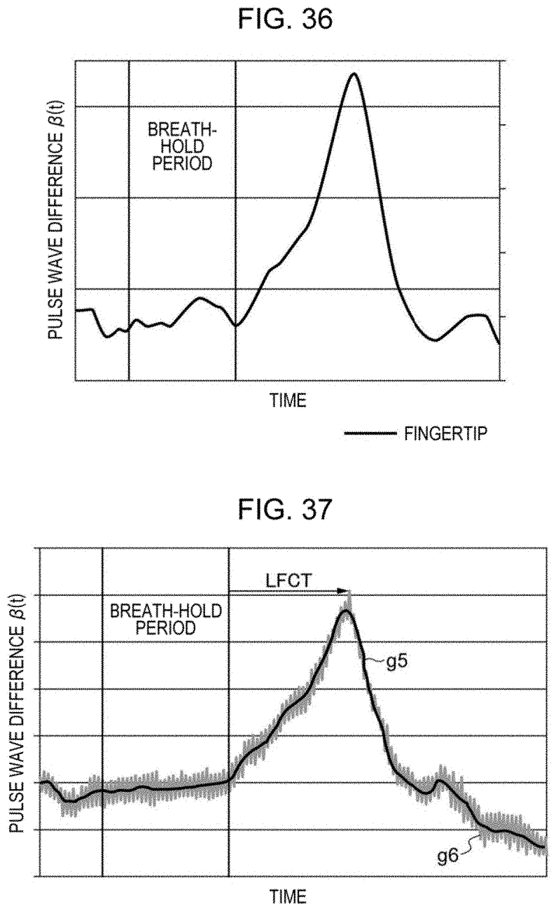

FIG. 36 is a graph illustrating an example of a monitor result by the pulse wave difference according to the present exemplary embodiment. In FIG. 36, the vertical axis represents the pulse wave difference β(t), while the horizontal axis represents time. As demonstrated by FIG. 36, the change in oxygen saturation due to a breath-hold is exhibited distinctly.

Subsequently, in step S114, the CPU 1020A calculates the pulse wave difference β(t) according to Formula (7) above on the basis of α×IR(t) corrected in step S112 and Red(t). If Red(t) is corrected, the pulse wave difference β(t) may be calculated according to Formula (8) above.

Subsequently, in step S116, the CPU 1020A determines whether the value representing the degree of correlation between the IR light signal and the red light signal (e.g., the coefficient of determination R²) is less than the threshold (e.g., 0.8). If it is determined that the value representing the degree of correlation is less than the threshold (case of positive determination), it is determined that the pulse wave difference β(t) calculated in step S114 is appropriate, and the flow advances to step S118. If it is determined that the value representing the degree of correlation is greater than or equal to the threshold (case of negative determination), it is determined that the pulse wave difference β(t) calculated in step S114 is inappropriate, and the flow advances to step S122.

In step S118, the CPU 1020A detects the inflection point of the blood oxygen concentration associated with a change in the amount of oxygen inhaled by the test subject on the basis of the pulse wave difference β(t) calculated in step S114.

In step S120, the CPU 1020A specifies, as the LFCT, the time from the point in time at which the amount of oxygen inhaled by the test subject is changed until the inflection point detected in step S118 and ends the series of steps according to the biological information measurement program. Note that in the present exemplary embodiment, the process goes up to the specification of the LFCT, but in addition, Formula (6) above may be applied to the specified LFCT to calculate the cardiac output, which is an example of the output.

FIG. 37 is a graph illustrating an example of the LFCT specified on the basis of the pulse wave difference β(t) according to the present exemplary embodiment. In FIG. 37, the vertical axis represents the pulse wave difference β(t), while the horizontal axis represents time.

As illustrated in FIG. 37, the LFCT is the time from the point in time at which breathing resumes until the inflection point indicated by the maximum value of the pulse wave difference β(t) (=α×IR(t)−Red(t)).

Note that in FIG. 37, a graph g5 represents the pulse wave difference β(t) as a moving average of sample n data (in this example, n=64). Also, a graph g6 represents the pulse wave difference β(t) for a case where the coefficient α=0.3583. In this way, by treating the pulse wave difference β(t) as a moving average of sample n data, residual pulse wave components due to differences in blood oxygen concentration are removed, and a more accurate LFCT is obtained.

Also, the graph g5 and the graph g6 illustrated in FIG. 37 demonstrate that immediately after the breath-hold period ends and breathing is resumed, the value of the pulse wave difference β(t) rises, reaches a single peak, and then falls. Since the pulse wave difference β(t) rises as the blood oxygen concentration falls, the point in time of the peak is the state of the lowest blood oxygen concentration, and the inflection point where the pulse wave difference β(t) starting to fall indicates that oxygen is starting to be taken into the blood due to the resumption of breathing. Consequently, the time from the resumption of breathing up to the peak is specified as the LFCT.

On the other hand, in step S122, the CPU 1020A gives an alarm that recommends remeasurement and ends the series of steps according to the biological information measurement program. For example, the alarm is given by causing the display 1022 to display a message that recommends remeasurement in which a test subject breathes out sufficiently before a breath-hold.

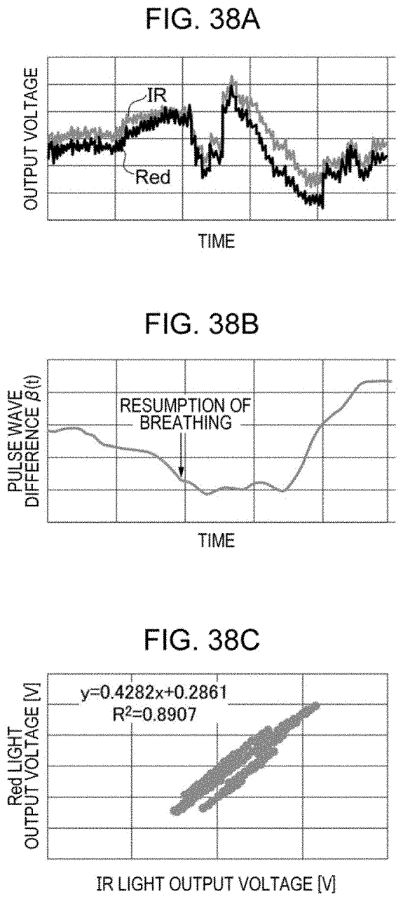

Next, referring to FIG. 38A to FIG. 41C, the correspondence relationship between plural inappropriate patterns (first to fourth inappropriate patterns) of the pulse wave difference β(t) and the coefficient of determination R² representing the degree of correlation between the IR light signal and the red light signal will specifically be described.

FIG. 38A is a graph illustrating time-series data of the IR light signal and the red light signal related to the first inappropriate pattern. FIG. 38B is a graph illustrating the first inappropriate pattern of the pulse wave difference β(t). FIG. 38C is a scatter diagram illustrating a correlation between the IR light output voltage and the red light output voltage with respect to the first inappropriate pattern.

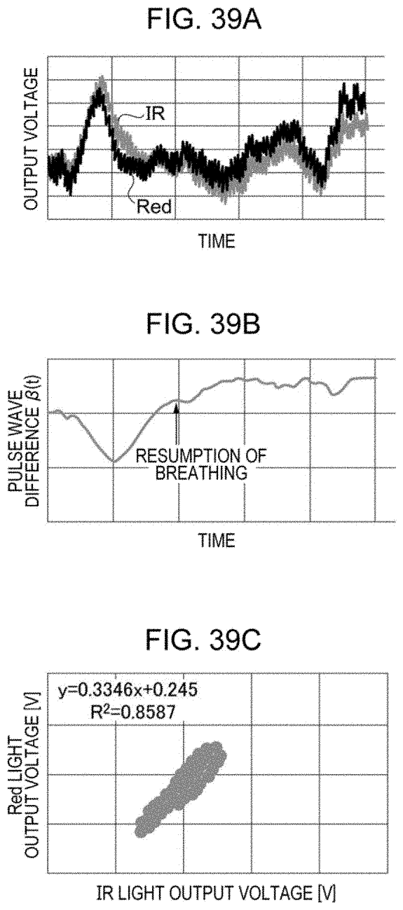

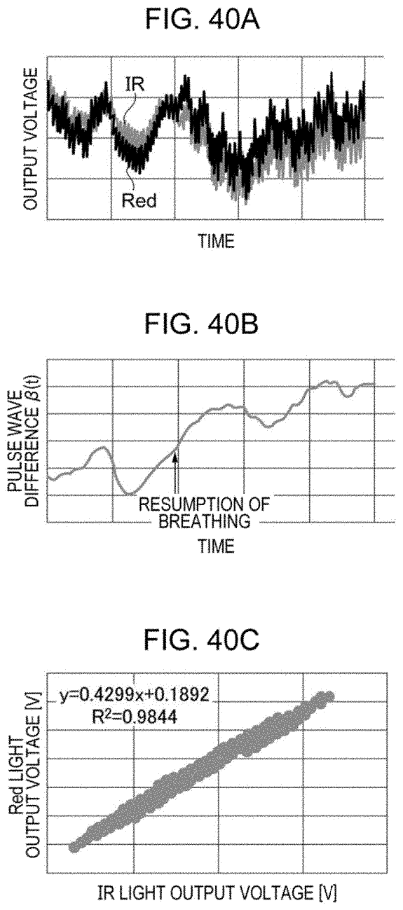

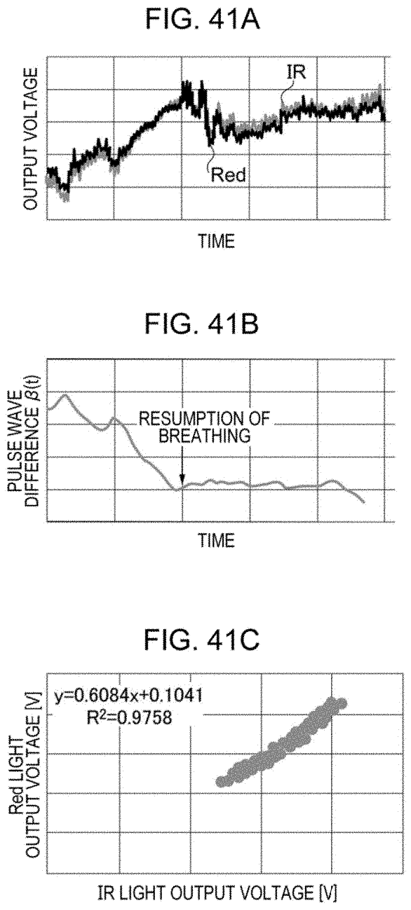

In the first inappropriate pattern, as illustrated in FIG. 38B, there are two or more minimum values, and the values are so close to each other that which one of them is to be determined to be the smallest. With an existing algorithm, if the difference between the smallest minimum value and the median is 1, and if a ratio with the difference between the second smallest minimum value and the median is greater than a threshold (0.5), the pattern has been determined to be the first inappropriate pattern. In contrast, as illustrated in FIG. 38C, the coefficient of determination R² representing the degree of correlation between the IR light signal and the red light signal is 0.8907, which is greater than or equal to the threshold (e.g., 0.8). In the method according to the present exemplary embodiment, if the coefficient of determination R² is greater than or equal to the threshold, the pulse wave difference β(t) is determined to be inappropriate, and thus, the first inappropriate pattern is determined in a simpler manner than with the existing algorithm. Note that the direction of a peak in FIG. 38B is reverse to the direction of a peak in FIG. 37. In addition, although the pulse wave difference β(t) is defined as α×IR(t)−Red(t) above, the pulse wave difference β(t) is defined as Red(t)−α×IR(t) here. The same applies to FIG. 39B, FIG. 40B, and FIG. 41B described below.

FIG. 39A is a graph illustrating time-series data of the IR light signal and the red light signal related to the second inappropriate pattern. FIG. 39B is a graph illustrating the second inappropriate pattern of the pulse wave difference β(t). FIG. 39C is a scatter diagram illustrating a correlation between the IR light output voltage and the red light output voltage with respect to the second inappropriate pattern.

In the second inappropriate pattern, as illustrated in FIG. 39B, although there is a minimum value, the pattern is broad. With an existing algorithm, if the largest value and the smallest value of the pulse wave difference β(t) after resumption of breathing are 1 and 0, respectively, and if the length of time during which both sides of the minimum and smallest value intersect with 0.2 is longer than or equal to a threshold (20 seconds), the pattern has been determined to be the second inappropriate pattern. In contrast, as illustrated in FIG. 39C, the coefficient of determination R² representing the degree of correlation between the IR light signal and the red light signal is 0.8587, which is greater than or equal to the threshold (e.g., 0.8). In the method according to the present exemplary embodiment, if the coefficient of determination R² is greater than or equal to the threshold, the pulse wave difference β(t) is determined to be inappropriate, and thus, the second inappropriate pattern is determined in a simpler manner than with the existing algorithm.

FIG. 40A is a graph illustrating time-series data of the IR light signal and the red light signal related to the third inappropriate pattern. FIG. 40B is a graph illustrating the third inappropriate pattern of the pulse wave difference β(t). FIG. 40C is a scatter diagram illustrating a correlation between the IR light output voltage and the red light output voltage with respect to the third inappropriate pattern.

In the third inappropriate pattern, as illustrated in FIG. 40B, the value of the pulse wave difference β(t) at the time of resumption of breathing is equivalent to or less than or equal to a minimum value. With an existing algorithm, as a result of comparison between the value at the time of resumption of breathing and the minimum and smallest value, if the value at the time of resumption of breathing is less than or equal to the minimum value, the pattern has been determined to be the third inappropriate pattern. In contrast, as illustrated in FIG. 40C, the coefficient of determination R² representing the degree of correlation between the IR light signal and the red light signal is 0.9844, which is greater than or equal to the threshold (e.g., 0.8). In the method according to the present exemplary embodiment, if the coefficient of determination R² is greater than or equal to the threshold, the pulse wave difference β(t) is determined to be inappropriate, and thus, the third inappropriate pattern is determined in a simpler manner than with the existing algorithm.

FIG. 41A is a graph illustrating time-series data of the IR light signal and the red light signal related to the fourth inappropriate pattern. FIG. 41B is a graph illustrating the fourth inappropriate pattern of the pulse wave difference β(t). FIG. 41C is a scatter diagram illustrating a correlation between the IR light output voltage and the red light output voltage with respect to the fourth inappropriate pattern.

In the fourth inappropriate pattern, as illustrated in FIG. 41B, the smallest value of the pulse wave difference β(t) after resumption of breathing is not a minimum value. With an existing algorithm, as a result of comparison between the smallest value of the pulse wave difference β(t) and the minimum and smallest value, if the smallest value and the minimum value do not correspond to each other, the pattern has been determined to be the fourth inappropriate pattern. In contrast, as illustrated in FIG. 41C, the coefficient of determination R² representing the degree of correlation between the IR light signal and the red light signal is 0.9758, which is greater than or equal to the threshold (e.g., 0.8). In the method according to the present exemplary embodiment, if the coefficient of determination R² is greater than or equal to the threshold, the pulse wave difference β(t) is determined to be inappropriate, and thus, the fourth inappropriate pattern is determined in a simpler manner than with the existing algorithm.

In the above manner, according to the present exemplary embodiment, a waveform pattern representing a change in the blood oxygen concentration is determined to be inappropriate if the correlation between two pulse waves is high, whereas a waveform pattern representing a change in the blood oxygen concentration is determined to be appropriate if the correlation is low. Thus, the inappropriate pattern is determined in a simpler manner than with the existing algorithm. In addition, since the state of the change in the blood oxygen concentration due to a breath-hold is obtained, if the change in the blood oxygen concentration is small, an alarm is given to recommend remeasurement, and thereby, the data reliability is improved.

### Fourth Exemplary Embodiment

In the third exemplary embodiment above, the coefficient α to be used for correction is the amplitude ratio of the amplitude of the IR light signal and the amplitude of the red light signal. In the present exemplary embodiment, the coefficient α is calculated from the inclination of a regression line obtained from the IR light signal and the red light signal.

Note that a biological information measurement apparatus according to the present exemplary embodiment includes the same structural elements as the biological information measurement apparatus 1010 described in the third exemplary embodiment above. Thus, a repeated description is omitted, and only a difference in the correction unit 1031 will be described with reference to FIG. 29.

The correction unit 1031 calculates the coefficient α from the inclination of a regression line obtained from the value of the IR light signal and the value of the red light signal.

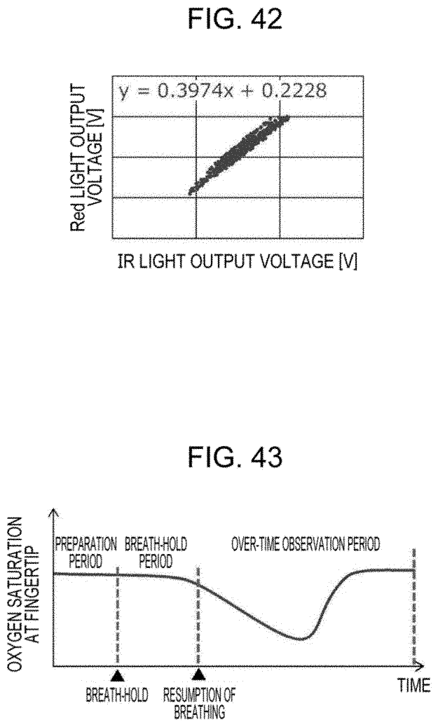

FIG. 42 is a scatter diagram illustrating an example of a correlation between the IR light output voltage and the red light output voltage according to the fourth exemplary embodiment.

From the scatter diagram in FIG. 42, a regression line is obtained. In this case, the regression line is obtained as y=0.3974x+0.2228. The inclination of the regression line “0.3974” is used as the coefficient α. In this case, if the number of heartbeats of the living body 8 is one, a low correlation may be unlikely to be obtained even if there is a change in the blood oxygen concentration. Thus, the regression line is desirably calculated from the value of the IR light signal and the value of the red light signal in a period of two or more heartbeats of the living body 8.

In addition, if the value representing the degree of correlation between the IR light signal and the red light signal (e.g., the coefficient of determination R²) is greater than or equal to a predetermined value (e.g., 0.9), the correction unit 1031 may calculate the coefficient α.

FIG. 43 illustrates an example of a preparation period, a breath-hold period, and an over-time observation period in an LFCT measurement.

In FIG. 43, the coefficient α is calculated from data of a regression line during a period before the LFCT measurement starts, in other words, before a breath-hold starts. During the period before a breath-hold starts, a change in the blood oxygen concentration is comparatively small, and the correlation between the IR light signal and the red light signal is high. For a test subject for whom the LFCT is comparatively short, the blood oxygen concentration starts to fall immediately after a breath-hold starts. For example, for a test subject for whom the LFCT is 10 seconds, a change in the blood oxygen concentration appears at about 10 seconds after a breath-hold starts. In this case, if the breath-hold period lasts 20 seconds, the blood oxygen concentration changes during half of the period. In contrast, not a large change in the blood oxygen concentration appears before a breath-hold starts. Thus, the coefficient α is calculated from data of a regression line during a period before a breath-hold starts.

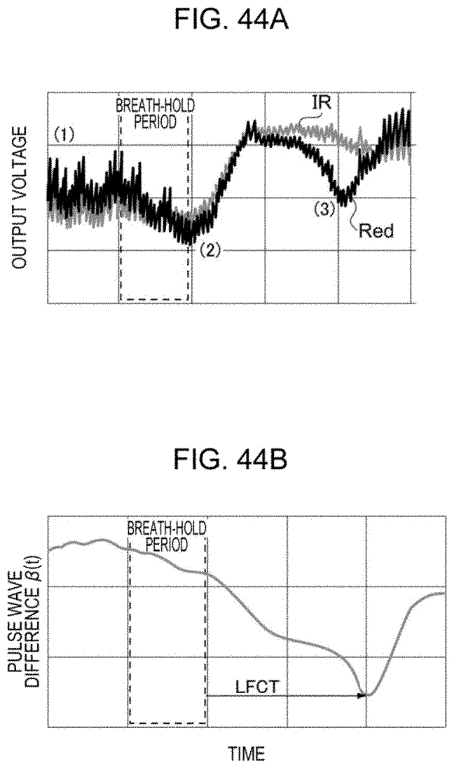

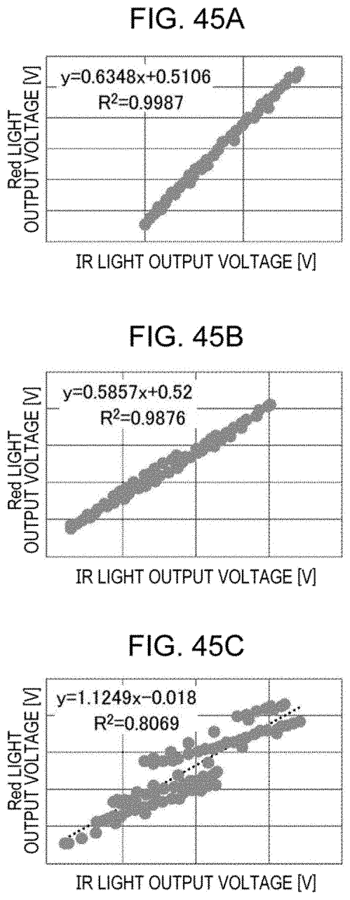

FIG. 44A, FIG. 44B, FIG. 45A, FIG. 45B, and FIG. 45C are graphs for describing the correlation between the IR light signal and the red light signal. FIG. 44A illustrates time-series data of the IR light signal and the red light signal. FIG. 44B illustrates the LFCT of the pulse wave difference β(t). FIG. 45A, FIG. 45B, and FIG. 45C are scatter diagrams illustrating the correlation between the IR light output voltage and the red light output voltage during respective periods in the time-series data of the IR light signal and the red light signal illustrated in FIG. 44A.

In FIG. 44A, the correlation between the IR light signal and the red light signal in a region (1) is comparatively high as the coefficient of determination R² illustrated in FIG. 45A is 0.9987. In this case, a change in the blood oxygen concentration is small, and the correlation of the regression line is high, and thus, the coefficient α may be determined in the region (1). Thus, an error of the coefficient α due to the change in the blood oxygen concentration is removed.

Similarly, in FIG. 44A, the correlation between the IR light signal and the red light signal in a region (2) is comparatively high as the coefficient of determination R² illustrated in FIG. 45B is 0.9876. In the region (2), the amount of blood changes. In this case, a change in the blood oxygen concentration is small, and the correlation of the regression line is high, and thus, the coefficient α may be determined in the region (2). Thus, an error of the coefficient α due to the change in the blood oxygen concentration is removed.

On the other hand, in FIG. 44A, the correlation between the IR light signal and the red light signal in a region (3) is comparatively low as the coefficient of determination R² illustrated in FIG. 45C is 0.8069. The region (3) is in the vicinity of a peak of the LFCT. For example, the coefficient α is desirably determined before a measurement starts (before the preparation period). In this case, on the basis of the determined coefficient α, a change in the blood oxygen concentration can be measured in real time. Specifically, when a change in the blood oxygen concentration is small, in other words, when the correlation is high, the coefficient α is determined. If the coefficient of determination is such that the correlation is one in the region (1) or (2), the coefficient α may be determined; if the coefficient of determination is such that the correlation is one in the region (3), the coefficient α is not determined.

The correction unit 1031 may further separate the regression line into a contraction period and an expansion period and may calculate the coefficient α if a difference between inclinations of the regression lines is within a predetermined range (e.g., 20%).

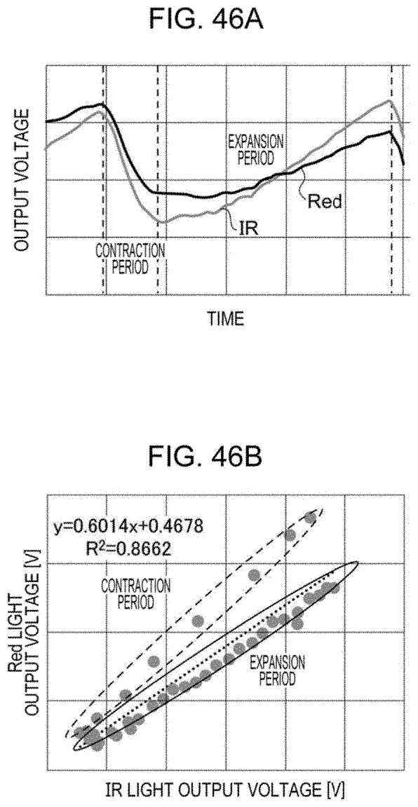

FIG. 46A is a graph illustrating time-series data of the IR light signal and the red light signal during the contraction period and the expansion period. FIG. 46B is a scatter diagram illustrating a correlation between the IR light output voltage and the red light output voltage during the contraction period and the expansion period. Data in FIG. 46B illustrates data of a single heartbeat when the blood oxygen concentration changes. In this case, there is a difference between the inclination of the regression line during the contraction period and the inclination of the regression line during the expansion period, and the correlation is low as a whole. Note that the contraction period is a period during which the heart contracts to pump out blood and the blood pressure increases, whereas the expansion period is a period during which the heart expands to receive blood running through the entire body and the blood pressure decreases.

As illustrated in FIG. 46B, if the blood oxygen concentration changes, a difference occurs between the inclination of the regression line during the contraction period and the inclination of the regression line during the expansion period. In other words, if the difference between the inclination of the regression line during the contraction period and the inclination of the regression line during the expansion period is small, since the blood oxygen concentration does not change, the correlation is high; if the difference is large, since the blood oxygen concentration changes, the correlation is low.

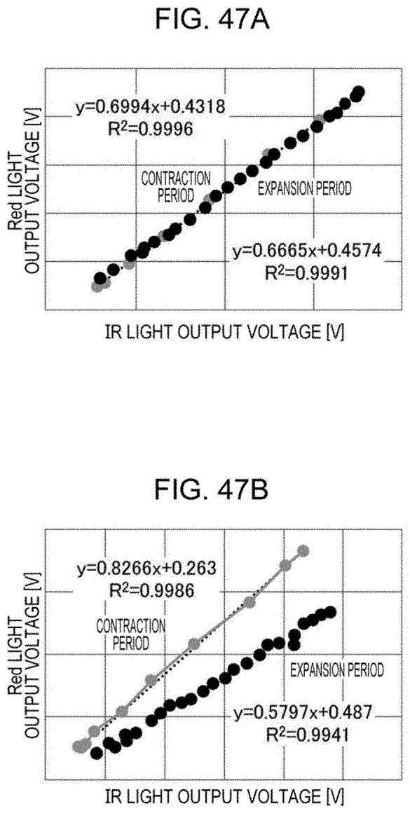

FIG. 47A is a scatter diagram illustrating an example of regression lines during the contraction period and the expansion period in a case where the blood oxygen concentration does not change. FIG. 47B is a scatter diagram illustrating an example of regression lines during the contraction period and the expansion period in a case where the blood oxygen concentration changes.

The scatter diagram in FIG. 47A illustrates an example of a case where the blood oxygen concentration does not change, and the inclination of the regression line during the contraction period is substantially the same as the inclination of the regression line during the expansion period. Note that the regression line during the contraction period is expressed as y=0.6994x+0.4318, whereas the regression line during the expansion period is expressed as y=0.6665x+0.4574. On the other hand, the scatter diagram in FIG. 47B illustrates an example of a case where the blood oxygen concentration changes, and the inclination of the regression line during the contraction period is different from the inclination of the regression line during the expansion period. Note that the regression line during the contraction period is expressed as y=0.8266x+0.263, whereas the regression line during the expansion period is expressed as y=0.5797x+0.487. Thus, as described above, if the difference between the inclinations of the regression lines is within a predetermined range (e.g., 20%), the coefficient α is calculated. Thus, an error of the coefficient α due to the change in the blood oxygen concentration is removed.

The correction unit 1031 may also calculate the coefficient α if a low frequency/high frequency (LF/HF) ratio, which is an indicator indicating a state of tension of the living body 8, is less than or equal to a threshold (e.g., 4.0).

If the living body 8 is in a state of tension, sympathetic nerves act to increase the heartbeat and cause peripheral vasoconstriction. Thus, the blood circulation state changes, which may influence an LFCT measurement. Thus, the measurement is desirably performed while the test subject remains in a resting state.

An example of an indicator for obtaining the state of tension is an LF/HF ratio, which is an integral ratio of low-frequency components (most of which originate from Mayer waves) and high-frequency components (most of which originate from breathing) of pulse waves. For example, if the LF/HF ratio is less than or equal to 4.0, the coefficient α may be determined. Using this indicator may prevent a measurement from being performed in a state where the neural activity is increased.

Cases have been described above where the coefficient α is determined if one of the conditions for the correlation between the IR light signal and the red light signal (correlation between pulse waves), the difference between inclinations of regression lines during the contraction period and the expansion period, and the LF/HF ratio is satisfied. The coefficient α may be determined if all of these conditions are satisfied.

In this manner, according to the present exemplary embodiment, a stable coefficient α is obtained from a large amount of data even if the pulsation is small. In addition, since the coefficient α is obtained from pulse wave data, a stable coefficient α is obtained even during a short period. Furthermore, by using the correlation between pulse waves, the difference between inclinations of regression lines during the contraction period and the expansion period, and the LF/HF ratio when determining the coefficient α, a stable coefficient α is obtained.

The above describes a biological information measurement apparatus according to the exemplary embodiments as an example. An exemplary embodiment may also be configured as a program causing a computer to execute the functions of each component provided in the biological information measurement apparatus. An exemplary embodiment may also be configured as a non-transitory computer readable medium storing the program.

Other configuration of the biological information measurement apparatus described in the exemplary embodiments above is an example and may be modified according to circumstances without departing from the gist.

Also, the process flow of the program described in the exemplary embodiments above is an example, and unnecessary steps may be removed, new steps may be added, or the processing sequence may be rearranged without departing from the gist.

Also, the exemplary embodiments above describe a case where the process according to the exemplary embodiments is implemented by a software configuration using a computer by executing a program, but the configuration is not limited thereto. An exemplary embodiment may also be implemented by a hardware configuration, or by a combination of a hardware configuration and a software configuration, for example.

The third exemplary embodiment and the fourth exemplary embodiment described above may be implemented as in the following aspects, for example.

A biological information measurement apparatus according to a first aspect includes a processor configured to: obtain a first signal representing a change in an amount of light of a first wavelength detected from a living body and a second signal representing a change in an amount of light of a second wavelength detected from the living body; correct either one of a value of the first signal and a value of the second signal by multiplying a corresponding one of the value of the first signal and the value of the second signal by a coefficient to reduce a difference between an amount of change in the first signal and an amount of change in the second signal associated with a change in an amount of arterial blood in the living body; calculate a waveform pattern representing a change in a blood oxygen concentration in the living body, the waveform pattern being represented as a difference between the value of the first signal and the value of the second signal, either of which is corrected by using the coefficient; and determine that the waveform pattern is appropriate if a value representing a degree of correlation between the first signal and the second signal is less than a threshold.

In a biological information measurement apparatus according to a second aspect, in the first aspect, the processor is configured to detect an inflection point of the blood oxygen concentration associated with a change in an amount of oxygen inhaled by the living body on the basis of a waveform pattern that the processor determines to be appropriate and specify a time from a point in time at which the amount of oxygen inhaled by the living body is changed until the detected inflection point of the blood oxygen concentration.

In a biological information measurement apparatus according to a third aspect, in the first or second aspect, the processor is configured to, if the value representing the degree of correlation is greater than or equal to the threshold, determine that the waveform pattern is inappropriate and gives an alarm that recommends remeasurement.

In a biological information measurement apparatus according to a fourth aspect, in the third aspect, the alarm includes a message that recommends remeasurement in which the living body breathes out sufficiently before a breath-hold.

In a biological information measurement apparatus according to a fifth aspect, in any of the first to fourth aspects, the value representing the degree of correlation is calculated from the first signal and the second signal during a predetermined period after an amount of oxygen inhaled by the living body is changed.

In a biological information measurement apparatus according to a sixth aspect, in the fifth aspect, the predetermined period is a period of greater than or equal to two heartbeats of the living body.

In a biological information measurement apparatus according to a seventh aspect, in any one of the first to sixth aspects, the coefficient is represented by an amplitude ratio of an amplitude of the first signal and an amplitude of the second signal before an amount of oxygen inhaled by the living body is changed, and the correction is performed by multiplying the value of the first signal or the value of the second signal by the coefficient after the amount of oxygen inhaled by the living body is changed.

In a biological information measurement apparatus according to an eighth aspect, in the first aspect, the processor is configured to calculate the coefficient from inclinations of regression lines obtained from the value of the first signal and the value of the second signal.

In a biological information measurement apparatus according to a ninth aspect, in the eighth aspect, the regression lines are obtained from the value of the first signal and the value of the second signal during a period of two or more heartbeats of the living body.

In a biological information measurement apparatus according to a tenth aspect, in the eighth or ninth aspect, the processor is configured to calculate the coefficient if the value representing the degree of correlation between the first signal and the second signal is greater than or equal to a predetermined value.

In a biological information measurement apparatus according to an eleventh aspect, in any one of the eighth to tenth aspects, the processor is configured to calculate the coefficient if the regression lines are separated into a contraction period and an expansion period, and a difference between the inclinations of the regression lines in the contraction period and the expansion period is within a predetermined range.

In a biological information measurement apparatus according to a twelfth aspect, in any one of the eighth to eleventh aspects, the processor is configured to calculate the coefficient if a low frequency/high frequency (LF/HF) ratio, which is an indicator indicating a state of tension of the living body, is less than or equal to a threshold.

A non-transitory computer readable medium storing a program according to a thirteenth aspect causes a computer to execute a process for biological information measurement, the process including: obtaining a first signal representing a change in an amount of light of a first wavelength detected from a living body and a second signal representing a change in an amount of light of a second wavelength detected from the living body; correcting either one of a value of the first signal and a value of the second signal by multiplying a corresponding one of the value of the first signal and the value of the second signal by a coefficient to reduce a difference between an amount of change in the first signal and an amount of change in the second signal associated with a change in an amount of arterial blood in the living body; calculating a waveform pattern representing a change in a blood oxygen concentration in the living body, the waveform pattern being represented as a difference between the value of the first signal and the value of the second signal, either of which is corrected by using the coefficient; and determining that the waveform pattern is appropriate if a value representing a degree of correlation between the first signal and the second signal is less than a threshold.

In the embodiments above, the term “processor” refers to hardware in a broad sense. Examples of the processor include general processors (e.g., CPU: Central Processing Unit) and dedicated processors (e.g., GPU: Graphics Processing Unit, ASIC: Application Specific Integrated Circuit, FPGA: Field Programmable Gate Array, and programmable logic device).

In the embodiments above, the term “processor” is broad enough to encompass one processor or plural processors in collaboration which are located physically apart from each other but may work cooperatively. The order of operations of the processor is not limited to one described in the embodiments above, and may be changed.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. A biological information measurement apparatus comprising:
a processor configured to:
if a predetermined number of a plurality of measurements of an oxygen circulation time are to be performed, before a predetermined oxygen-circulation-time measurement period ends during a first measurement of the oxygen circulation time,
notify a test subject of a breath-hold instruction as a preparation for a second measurement of the oxygen circulation time, the test subject being a person for whom the oxygen circulation time is measured, the second measurement being a subsequent measurement to the first measurement.

2. The biological information measurement apparatus according to claim 1, wherein the processor is configured to vary a breathing adjustment period in accordance with a measurement condition of the oxygen circulation time of the test subject, the breathing adjustment period being a period from when the test subject who is instructed to resume breathing after a breath-hold until the test subject is instructed to hold breath again for the second measurement of the oxygen circulation time.

3. The biological information measurement apparatus according to claim 2, wherein the processor is configured to control the breathing adjustment period such that the breathing adjustment period becomes longer as a measurement of the oxygen circulation time of the test subject approaches a final measurement.

4. The biological information measurement apparatus according to claim 2, wherein the processor is configured to perform control such that the breathing adjustment period corresponds to a period from when the test subject who is instructed to resume breathing after a breath-hold until an inflection point at which oxygen saturation in blood of the test subject turns from a decrease to an increase is detected in association with resumption of breathing of the test subject.

5. The biological information measurement apparatus according to claim 4, wherein the processor is configured to perform control such that measuring the oxygen circulation time is continued until the inflection point is detected during a final measurement of the oxygen circulation time of the test subject.

6. The biological information measurement apparatus according to claim 1, wherein the processor is configured to control a breath-hold period such that the breath-hold period becomes shorter as a measurement of the oxygen circulation time of the test subject approaches a final measurement, the breath-hold period being a period from when the test subject is instructed to hold breath until the test subject is instructed to resume breathing.

7. The biological information measurement apparatus according to claim 2, wherein the processor is configured to control a breath-hold period such that the breath-hold period becomes shorter as a measurement of the oxygen circulation time of the test subject approaches a final measurement, the breath-hold period being a period from when the test subject is instructed to hold breath until the test subject is instructed to resume breathing.

8. The biological information measurement apparatus according to claim 3, wherein the processor is configured to control a breath-hold period such that the breath-hold period becomes shorter as a measurement of the oxygen circulation time of the test subject approaches a final measurement, the breath-hold period being a period from when the test subject is instructed to hold breath until the test subject is instructed to resume breathing.

9. The biological information measurement apparatus according to claim 4, wherein the processor is configured to control a breath-hold period such that the breath-hold period becomes shorter as a measurement of the oxygen circulation time of the test subject approaches a final measurement, the breath-hold period being a period from when the test subject is instructed to hold breath until the test subject is instructed to resume breathing.

10. The biological information measurement apparatus according to claim 5, wherein the processor is configured to control a breath-hold period such that the breath-hold period becomes shorter as a measurement of the oxygen circulation time of the test subject approaches a final measurement, the breath-hold period being a period from when the test subject is instructed to hold breath until the test subject is instructed to resume breathing.

11. The biological information measurement apparatus according to claim 1, wherein the processor is configured to vary a breath-hold period in accordance with a difference between a minimum value of oxygen saturation in blood of the test subject and a predetermined reference value of oxygen saturation prescribing a minimum value of oxygen saturation necessary to measure the oxygen circulation time, the breath-hold period being a period from when the test subject is instructed to hold breath until the test subject is instructed to resume breathing.

12. The biological information measurement apparatus according to claim 2, wherein the processor is configured to vary a breath-hold period in accordance with a difference between a minimum value of oxygen saturation in blood of the test subject and a predetermined reference value of oxygen saturation prescribing a minimum value of oxygen saturation necessary to measure the oxygen circulation time, the breath-hold period being a period from when the test subject is instructed to hold breath until the test subject is instructed to resume breathing.

13. The biological information measurement apparatus according to claim 3, wherein the processor is configured to vary a breath-hold period in accordance with a difference between a minimum value of oxygen saturation in blood of the test subject and a predetermined reference value of oxygen saturation prescribing a minimum value of oxygen saturation necessary to measure the oxygen circulation time, the breath-hold period being a period from when the test subject is instructed to hold breath until the test subject is instructed to resume breathing.

14. The biological information measurement apparatus according to claim 4, wherein the processor is configured to vary a breath-hold period in accordance with a difference between a minimum value of oxygen saturation in blood of the test subject and a predetermined reference value of oxygen saturation prescribing a minimum value of oxygen saturation necessary to measure the oxygen circulation time, the breath-hold period being a period from when the test subject is instructed to hold breath until the test subject is instructed to resume breathing.

15. The biological information measurement apparatus according to claim 5, wherein the processor is configured to vary a breath-hold period in accordance with a difference between a minimum value of oxygen saturation in blood of the test subject and a predetermined reference value of oxygen saturation prescribing a minimum value of oxygen saturation necessary to measure the oxygen circulation time, the breath-hold period being a period from when the test subject is instructed to hold breath until the test subject is instructed to resume breathing.

16. The biological information measurement apparatus according to claim 11, wherein the processor is configured to make the breath-hold period shorter as the difference is larger if the minimum value is less than or equal to the reference value and makes the breath-hold period longer as the difference is larger if the minimum value exceeds the reference value.

17. The biological information measurement apparatus according to claim 12, wherein the processor is configured to make the breath-hold period shorter as the difference is larger if the minimum value is less than or equal to the reference value and makes the breath-hold period longer as the difference is larger if the minimum value exceeds the reference value.

18. The biological information measurement apparatus according to claim 13, wherein the processor is configured to make the breath-hold period shorter as the difference is larger if the minimum value is less than or equal to the reference value and makes the breath-hold period longer as the difference is larger if the minimum value exceeds the reference value.

19. A non-transitory computer readable medium storing a program causing a computer to execute a process for biological information measurement, the process comprising:
if a predetermined number of a plurality of measurements of an oxygen circulation time are to be performed, before a predetermined oxygen-circulation-time measurement period ends during a first measurement of the oxygen circulation time,
notifying a test subject of a breath-hold instruction as a preparation for a second measurement of the oxygen circulation time, the test subject being a person for whom the oxygen circulation time is measured, the second measurement being a subsequent measurement to the first measurement.

20. A biological information measurement apparatus comprising:
a processor configured to:
obtain a first signal representing a change in an amount of light of a first wavelength detected from a living body and a second signal representing a change in an amount of light of a second wavelength detected from the living body;
correct either one of a value of the first signal and a value of the second signal by multiplying a corresponding one of the value of the first signal and the value of the second signal by a coefficient to reduce a difference between an amount of change in the first signal and an amount of change in the second signal associated with a change in an amount of arterial blood in the living body;
calculate a waveform pattern representing a change in a blood oxygen concentration in the living body, the waveform pattern being represented as a difference between the value of the first signal and the value of the second signal, either of which is corrected by using the coefficient; and
determine that the waveform pattern is appropriate if a value representing a degree of correlation between the first signal and the second signal is less than a threshold.
